SEQUENCE LISTING (10 sequences)

SEQ ID NO: 1 - protein, 10 aa, synthetic construct
GYTFTSYAMH

SEQ ID NO: 2 - protein, 17 aa, synthetic construct
YINPYNDVSRYNEKFQG

SEQ ID NO: 3 - protein, 12 aa, synthetic construct
EARPTSAPYFDV

SEQ ID NO: 4 - protein, 17 aa, synthetic construct
KSSQSLLNSRTRKNYLA

SEQ ID NO: 5 - protein, 7 aa, synthetic construct
WTSTRES

SEQ ID NO: 6 - protein, 8 aa, synthetic construct
KQSYNLFT

SEQ ID NO: 7 - protein, 119 aa, synthetic construct
EVQLQQSGPELVKPGASVKMSCKASGYTFTSYAMHWVKQKPGQGLEWIGYINPYNDVSRYNEKFQGKATLTSDKSSNTAYMELSSLTSEDSAVYYCEARPTSAPYFDVFGTGTTVTVSS

SEQ ID NO: 8 - protein, 113 aa, synthetic construct
DIVMSQSPSSLAASVGEKVTMTCKSSQSLLNSRTRKNYLAWYQQKPGQSPKLLIYWTSTRESGVPDRFTGSGSGTDFTLTISSVQTEDLAIYYCKQSYNLFTFGSGTKLEIKR

SEQ ID NO: 9 - DNA, 357 bp, synthetic construct
  source 1..357: mol_type = other DNA
gaggtccagctgcagcagtctggacctgaactggtaaagcctggggcttcagtgaagatgtcctgcaaggcttctggatacacattcactagctatgctatgcactgggtgaagcagaagcctgggcagggccttgagtggattggatatattaatccttacaatgatgttagtaggtacaatgagaagttccaaggcaaggccacactgacttcagacaaatcctccaacacagcctacatggagctcagcagcctgacctctgaggactctgcggtctattactgtgaggcccgccctacttcggctccgtacttcgatgtctttggcacagggaccacggtcaccgtctcctca

SEQ ID NO: 10 - DNA, 339 bp, synthetic construct
  source 1..339: mol_type = other DNA
gacattgtgatgtcacagtctccatcctccctggctgcgtcagtgggagagaaggtcactatgacctgcaaatccagtcagagtctgctcaacagtagaacccgaaagaactacttggcttggtatcagcagaaaccagggcagtctcctaaactgctgatctactggacatccactagggaatctggggtccctgatcgcttcacaggcagtggatctgggacagatttcactctcaccatcagcagtgtgcagactgaagacctggcaatttattactgcaagcaatcttataatctattcacgttcggctcggggacaaagttggaaataaaacgg

(19) United States
(12) Patent Application Publication
(10) Pub. No.: US 2026/0007767 A1
(43) Pub. Date: Jan. 8, 2026

(54) ANTI-SCUBE1 ANTIBODY HAVING HIGH INTERNALIZATION CAPACITY IN LEUKEMIA

(71) Applicant: ACADEMIA SINICA, Taipei (TW)

(72) Inventors: Ruey-Bing YANG, Taipei (TW); Yuh-Charn LIN, Taichung (TW); Binay Kumar SAHOO, Odisha (IN)

(21) Appl. No.: 18/881,174

(22) PCT Filed: Jul. 5, 2023
(86) PCT No.: PCT/US23/69615

§ 371 Date: Jan. 3, 2025

Related U.S. Application Data
(60) Provisional application No. 63/367,696, filed on Jul. 5, 2022.

(51) Int. Cl. A61K 47/68; A61P 35/02; C07K 16/28

(52) U.S. Cl. CPC A61K 47/6849; A61K 47/68031; A61P 35/02; C07K 16/28; C07K 2317/77

(57) ABSTRACT

The present disclosure relates to anti-SCUBE1 antibodies, antigen-binding fragments thereof, and antibody drug conjugates (ADCs), as well as methods of treating and/or preventing SCUBE1-expressing cancers.

### PRIORITY INFORMATION

The subject application is a 371 National Stage of International Patent Application No. PCT/US23/69615, filed Jul. 5, 2023, which claims benefit of and priority to U.S. Provisional Patent Application No. 63/367,696, filed Jul. 5, 2022, the contents of which are incorporated in their entirety by reference herewith.

### SEQUENCE LISTING INFORMATION

The instant application contains a Sequence Listing which has been filed electronically in XML format and is hereby incorporated by reference in its entirety. Said XML copy, created on Jul. 4, 2023 and is named “G4590-16700NP_SeqListing_20250103.xml” and is 11 kilobytes in size.

### FIELD OF THE INVENTION

The present disclosure relates to anti-SCUBE1 antibodies, antibody drug conjugates (ADCs), and antigen-binding fragments thereof, as well as methods of treating and/or preventing SCUBE1-expressing cancers.

### BACKGROUND OF THE INVENTION

SCUBE1 (signal peptide-CUB-EGF-like repeat-containing protein) is the founding member of the SCUBE (signal peptide-CUB-EGF-like repeat-containing protein) family membrane anchorage protein. SCUBE1 is highly expressed on membrane of leukemia cells and critical for its survival. It has 5 distinctive protein domains including an amino-terminal signal sequence, 9 tandem copies of EGF-like repeats, a spacer region, 3 cysteine-rich (CR) motifs, and 1 CUB domain at the carboxy-terminus.

The function of SCUBE proteins largely depends on their subcellular distribution and cell-type-specific expression. For instance, SCUBE1 is produced and stored in the α-granules of resting platelets. Upon pathological stimulation, it translocates from α-granules to the platelet surface where it is proteolytically released and incorporated into a thrombus.

Apart from secretion, SCUBE proteins are also expressed as peripheral membrane proteins tethered on the cell surface via the spacer and CR repeats by two independent mechanisms (i.e., electrostatic and lectin-glycan interactions, respectively), where they function as co-receptors in promoting the signaling activity of numerous growth factors mediated by receptor tyrosine kinases (TKs) or receptor serine/threonine kinases including fibroblast growth factor receptor (FGFR), vascular endothelial growth factor receptor (VEGFR), and bone morphogenetic protein receptor (BMPR). Moreover, SCUBE2 interacting with VEGFR2 on the cell surface could be internalized by a monoclonal anti-SCUBE2 antibody to inhibit VEGF-stimulated tumor angiogenesis, thus suppressing the pathological growth of solid tumors originating from the lung, pancreas, colon, melanoma, or Leydig cells.

### SUMMARY OF THE INVENTION

Described herein are antibodies, and antigen binding portions thereof, that recognize the EGF-like repeats #1-3 of SCUBE1 and surprisingly exhibit a high internalization capacity, as well as compositions and methods of using said antibodies. In particular, the antibodies and fragments described herein can be used in anti-SCUBE1 antibody-drug conjugates (ADCs) for killing SCUBE1-expressing cancer cells. Particularly, the present disclosure first shows that SCUBE1 is a direct target of HOXA9/MEIS1 that is highly expressed on the MLL-r cell surface and predicts poor prognosis in de novo AML.

In some embodiments, the disclosure provides an antibody or antigen-binding fragment thereof that specifically binds to SCUBE1; wherein the antibody or antigen-binding fragment thereof comprises a heavy chain variable region and/or a light chain variable region, wherein:
the heavy chain variable region comprises:
   a complementary determining region (CDR) sequence CDRH1 comprising the amino acid sequence of GYTFTSYAMH (SEQ ID NO: 1) or an amino acid sequence having at least 80% sequence identity to SEQ ID NO: 1;
   a CDRH2 sequence comprising the amino acid sequence of YINPYNDVSRYNEKFQG (SEQ ID NO: 2) or an amino acid sequence having at least 80% sequence identity to SEQ ID NO: 2; and
   a CDRH3 sequence comprising the amino acid sequence of EARPTSAPYFDV (SEQ ID NO: 3) or an amino acid sequence having at least 80% sequence identity to SEQ ID NO: 3; and wherein:
the light chain variable region comprises:
   a CDRL1 sequence comprising the amino acid sequence of KSSQSLLNSRTRKNYLA (SEQ ID NO: 4) or an amino acid sequence having at least 80% sequence identity to SEQ ID NO: 4;
   a CDRL2 sequence comprising the amino acid sequence of WTSTRES (SEQ ID NO: 5) or an amino acid sequence having at least 80% sequence identity to SEQ ID NO: 5; and
   a CDRL3 sequence comprising the amino acid sequence of KQSYNLFT (SEQ ID NO: 6) or an amino acid sequence having at least 80% sequence identity to SEQ ID NO: 6.

In one embodiment, the CDRH1 sequence comprises the amino acid sequence of GYTFTSYAMH (SEQ ID NO: 1) or an amino acid sequence having at least 80%, 90%, 95%, or 99% sequence identity to SEQ ID NO: 1. In one embodiment, the CDRH1 sequence comprises the amino acid sequence of GYTFTSYAMH (SEQ ID NO: 1). In one embodiment, the CDRH1 sequence consists of the amino acid sequence of GYTFTSYAMH (SEQ ID NO: 1).

In one embodiment, the CDRH2 sequence comprises the amino acid sequence of YINPYNDVSRYNEKFQG (SEQ ID NO: 2) or an amino acid sequence having at least 80%, 90%, 95%, or 99% sequence identity to SEQ ID NO: 2. In one embodiment, the CDRH2 sequence comprises the amino acid sequence of YINPYNDVSRYNEKFQG (SEQ ID NO: 2). In one embodiment, the CDRH2 sequence consists of the amino acid sequence of YINPYNDVSRYNEKFQG (SEQ ID NO: 2).

In one embodiment, the CDRH3 sequence comprises the amino acid sequence of EARPTSAPYFDV (SEQ ID NO: 3) or an amino acid sequence having at least 80%, 90%, 95%, or 99% sequence identity to SEQ ID NO: 3. In one embodiment, the CDRH3 sequence comprises the amino acid sequence of EARPTSAPYFDV (SEQ ID NO: 3). In one embodiment, the CDRH3 sequence consists of the amino acid sequence of EARPTSAPYFDV (SEQ ID NO: 3).

In one embodiment, the CDRL1 sequence comprises the amino acid sequence of KSSQSLLNSRTRKNYLA (SEQ ID NO: 4) or an amino acid sequence having at least 80%, 90%, 95%, or 99% sequence identity to SEQ ID NO: 4. In one embodiment, the CDRL1 sequence comprises the amino acid sequence of KSSQSLLNSRTRKNYLA (SEQ ID NO: 4). In one embodiment, the CDRL1 sequence consists of the amino acid sequence of KSSQSLLNSRTRKNYLA (SEQ ID NO: 4).

In one embodiment, the CDRL2 sequence comprises the amino acid sequence of WTSTRES (SEQ ID NO: 5) or an amino acid sequence having at least 80%, 90%, 95%, or 99% sequence identity to SEQ ID NO: 5. In one embodiment, the CDRL2 sequence comprises the amino acid sequence of WTSTRES (SEQ ID NO: 5). In one embodiment, the CDRL2 sequence consists of the amino acid sequence of WTSTRES (SEQ ID NO: 5).

In one embodiment, the CDRL3 sequence comprises the amino acid sequence of KQSYNLFT (SEQ ID NO: 6) or an amino acid sequence having at least 80%, 90%, 95%, or 99% sequence identity to SEQ ID NO: 6. In one embodiment, the CDRL3 sequence comprises the amino acid sequence of KQSYNLFT (SEQ ID NO: 6). In one embodiment, the CDRL3 sequence consists the amino acid sequence of KQSYNLFT (SEQ ID NO: 6).

In some embodiments of the disclosure, the antibody or antigen-binding fragment thereof comprises a heavy chain variable region comprising the amino acid sequence of SEQ ID NO: 7 or an amino acid sequence having at least 80% sequence identity to SEQ ID NO: 7; and/or a light chain variable region comprising the amino acid sequence of SEQ ID NO: 8 or an amino acid sequence having at least 80% sequence identity to SEQ ID NO: 8.

In one embodiment, the heavy chain variable region comprises the amino acid sequence of SEQ ID NO: 7 or an amino acid sequence having at least 80%, 90%, 95%, or 99% sequence identity to SEQ ID NO: 7. In one embodiment, the heavy chain variable region comprises the amino acid sequence of SEQ ID NO: 7. In one embodiment, the heavy chain variable region consists of the amino acid sequence of SEQ ID NO: 7.

In one embodiment, light chain variable region comprises the amino acid sequence of SEQ ID NO: 8 or an amino acid sequence having at least 80%, 90%, 95%, or 99% sequence identity to SEQ ID NO: 8. In one embodiment, light chain variable region comprises the amino acid sequence of SEQ ID NO: 8. In one embodiment, light chain variable region consists of the amino acid sequence of SEQ ID NO: 8.

In some embodiments of the disclosure, the antibody is a monoclonal antibody, chimeric antibody, humanized antibody or human antibody.

The present disclosure provides a vector encoding the antibody or antigen-binding fragment thereof as disclosed herein.

The present disclosure provides a genetically engineered cell expressing the antibody or antigen-binding fragment thereof as disclosed herein or containing the vector as disclosed herein.

The present disclosure provides a pharmaceutical composition comprising the antibody or antigen-binding fragment thereof as disclosed herein and pharmaceutically acceptable carrier and, optionally, a further therapeutic agent.

In another aspect, the disclosure provides an antibody-drug conjugate (ADC) comprising an anti-SCUBE1 antibody, or an antigen-binding fragment thereof, conjugated to a cytotoxin.

In some embodiments, the ADC has the structure of the following formula:

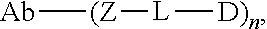

wherein an antibody or antigen-binding fragment thereof (Ab) is conjugated (covalently linked) to a linker (L), through a chemical moiety (Z), and further to a cytotoxin moiety (“drug,” D). n represents the number of drugs linked to the antibody.

In some embodiments, the cytotoxin is a microtubule-binding agent (for instance, maytansine or a maytansinoid), an amatoxin, Pseudomonas exotoxin A, deBouganin, diphtheria toxin, saporin, an auristatin, an anthracycline, a calicheamicin, irinotecan, SN-38, a duocarmycin, a pyrrolobenzodiazepine, a pyrrolobenzodiazepine dimer, an indolinobenzodiazepine, an indolinobenzodiazepine dimer, or a variant thereof. In one certain embodiment, the cytotoxin is monomethyl auristatin E (MMAE).

In one embodiment, n is about 1 to about 20.

In some embodiments, the cytotoxin is DNA-intercalating agents, (e.g., anthracyclines), agents capable of disrupting the mitotic spindle apparatus (e.g., Vinca alkaloids, maytansine, maytansinoids, and derivatives thereof), RNA polymerase inhibitors (e.g., an amatoxin, such as .alpha.-amanitin, and derivatives thereof), and agents capable of disrupting protein biosynthesis (e.g., agents that exhibit rRNA N-glycosidase activity, such as saporin and ricin A-chain).

In another aspect, the invention provides a method of treating and/or preventing a SCUBE1-expressing cancer in a subject, said method comprising administering an ADC described herein to the subject.

In some embodiments, the cancer is a blood cancer. In some embodiments, the cancer is leukemia; preferably, leukemia caused by MLL rearrangements. In some embodiments, the cancer is AML. In some embodiments, the AML is MLL-r AML.

In another aspect, the invention provides a pharmaceutical composition comprising any of the ADCs described herein, and a pharmaceutically acceptable carrier.

### BRIEF DESCRIPTION OF THE DRAWINGS

The subject application file contains at least one drawing executed in color. Copies of this patent or patent application publication with color drawing(s) will be provided by the U.S. Patent Office upon request and payment of the necessary fee. The following drawings form part of the present specification and are included to further demonstrate certain aspects of the present disclosure, the inventions of which can be better understood by reference to one or more of these drawings in combination with the detailed description of specific embodiments presented herein. Exemplary embodiments are illustrated in referenced figures. It is intended that the embodiments and figures disclosed herein are to be considered illustrative rather than restrictive.

FIGS. 1A-1F show expression of SCUBE1 in MLL-r AML and its association with prognosis of AML patients. FIG. 1A shows expression of SCUBE1 on surface of the leukemia or lymphoma cell lines determined by flow cytometry with anti-SCUBE1 monoclonal antibody (solid line) compared to corresponding isotype control antibody (dash line). Note that SCUBE1 is highly expressed in MLL-r (MLL-AF9 or MLL-AF4) AML including THP-1, NOMO-1, MOLM-13, and MV4-11 cells as well as Daudi (Burkitt's lymphoma) cells. FIG. 1B shows a summary of SCUBE1 expression in acute leukemia or lymphoma cell lines. FIGS. 1C and 1D show expression of SCUBE1 in AML cell lines bearing MLL-AF9 translocation determined at the mRNA level by qPCR (FIG. 1C) and protein level by western blot analysis (FIG. 1D). Anti-SCUBE1 monoclonal antibody (mAb) (#7) described previously was used for western blot and flow cytometry analyses. Data are mean±SD from 3 independent experiments. **p<0.01. FIG. 1E shows overall survival and FIG. 1E shows disease-free survival with SCUBE1 high (red line) and low (blue line) expression patient groups. Data were derived from GSE68469 and GSE71014 datasets.

FIGS. 2A-2H show that HOXA9 and MEIS1 bind and transactivate the regulatory elements of SCUBE1 in MLL-AF9 cells. FIG. 2A shows a graphical representation of predicted binding sites of HOXA9 and MEIS1 on human SCUBE1 regulatory regions. The binding sites of MEIS1 and HOXA9 on SCUBE1 enhancer or regulator regions were predicted by using the PROMO database. The overlapping binding sites of HOXA9 and MEIS1 were found in two regions: region 1 (upstream of SCUBE1 transcription start site) and region 2 (downstream of SCUBE1 transcription start site). FIG. 2B shows a ChIP assay of KG-1a, THP-1, or NOMO-1 cells with anti-MEIS1 antibody and enriched fragments analyzed by using RT-PCR. Oligonucleotide F1/R1 or F2/R2 primer pairs were used to amplify ˜400 bp of region 1 or 2 of enriched fragments, respectively. FIG. 2C shows a graphical Graphical illustration of luciferase-reporter constructs of SCUBE1 regulatory regions. The putative regulatory 440 bp of region 1 or 1389 bp of region 2 was cloned into the pGL3-basic vector. FIG. 2D shows a luciferase reporter assay with overexpression of HOXA9, MEIS1, or combined HOXA9 and MEIS1 together with the region 1 or 2 reporter constructs in HepG2 cells. Firefly luciferase activity was normalized to Renilla luciferase activity. FIGS. 2E and 2F show shRNA-mediated knockdown of transcription factors HOXA9 and MEIS1 at protein and mRNA levels in THP-1 or NOMO-1 cells. The quantified band intensities were normalized to loading controls and are mentioned below the corresponding bands. FIGS. 2G and 2H show mRNA and protein levels of SCUBE1 with HOXA9/MEIS1 knockdown in THP-1 and NOMO-1 cells. The quantified band intensities were normalized to loading controls and are mentioned below the corresponding bands. Data are mean±SD from 3 independent experiments. *P<0.05, **P<0.01.

FIGS. 3A-3D show inducible knockdown of SCUBE1 in MLL-AF9-translocated AML reduced cell growth and increased survival rate of mice. FIG. 3A shows a schematic representation of in vivo experiment to analyze the effect of SCUBE1 knockdown on the growth of THP-1 or NOMO-1 cells. Sub-lethal irradiation of NSG mice performed at day 0 followed by intravenous injection of THP-1 or NOMO-1 cells with inducible SCUBE1-shRNA #2 clone. Doxycycline was omitted or added in the drinking water for mice on day 1. Spleen and bone-marrow infiltration was measured on day 28, and survival rate was analyzed until all mice showed diseased symptoms (hunched back, lack of mobility, paralysis of hind limbs, ruffled coat). Mice were sacrificed on day 28 by cervical dislocation, and spleen and femur were isolated. FIG. 3B show that bone marrow was isolated from femur and human leukemic cell infiltration was measured by flow cytometry with anti-human CD45 antibody. FIG. 3C shows that spleen enlargement was measured by ratio of spleen weight to body weight. FIG. 3D shows a Kaplan-Meier curve showing survival of NSG mice engrafted with THP-1 or NOMO-1 cells bearing doxycycline-inducible SCUBE1-shRNA #2 clone and with (red line) or without (black line) doxycycline treatment. The median survival days were 45 days (−Dox) and 65 days (+Dox) or 42 days (−Dox) and 58 days (+Dox) for THP-1 and NOMO-1 cells, respectively. *P<0.05, **P<0.01.

FIGS. 4A-4E show that Scube1 is important for initiation of MLL-AF9 induced leukemia. FIG. 4A shows a schematic representation of experimental procedures to evaluate the role of SCUBE1 in initiation of leukemia. c-Kit⁺ hematopoietic cells were isolated from Scube1 KO or WT C57BL/6 mouse bone marrow, followed by transduction of MLL-AF9 retrovirus or SCUBE1 lentivirus and methylcellulose colony formation assay. After a third round of colony formation, cells were intravenously injected into sub-lethally irradiated C57BL/6 mice. When the primary transplanted mice showed symptoms of disease, leukemic cells were isolated from bone marrow and secondary transplantation was performed. FIG. 4B shows a methylcellulose colony formation assay after three rounds of replating after MLL-AF9 transduction. FIG. 4C shows spleen enlargement of secondary transplanted mice. FIG. 4D show H&E-stained spleen histology of secondary transplanted mice. Images were acquired with an Olympus microscope equipped with an Olympus DP70 digital camera; Original magnification 10×; scale bar=200 μm. FIG. 4E shows a Kaplan-Meier curve showing survival of secondary transplanted mice. The median survival for WT (black line), KO (red line), and KO+SCUBE1 (blue line) cells was 96.5, 190, and 143 days, respectively. Data are mean±SD from 3 independent experiments. *P<0.05, **P<0.01.

FIGS. 5A-5E show that Scube1 is critical for maintaining MLL-AF9-transformed leukemia stem cells. FIG. 5A shows a representation of experimental procedures to evaluate the role of Scube1 in maintaining leukemia stem cell in vitro. c-Kit⁺ hematopoietic cells were isolated from Scube1^{f/f} or Scube1^{f/f}; R26^{CreERT2} C57BL/6 mouse bone marrow, followed by transduction of MLL-AF9 retrovirus and three rounds of methylcellulose colony formation assay. At the fourth round, 4-OHT (4-hydroxy tamoxifen) 30 nM was added for the Cre-mediated Scube1 knockout. FIG. 5B shows a methylcellulose colony formation assay at the fourth round after 4-OHT treatment. FIG. 5C shows a schematic representation of experimental procedure to examine the role of Scube1 in maintenance of leukemia stem cells in vivo. c-Kit⁺ hematopoietic cells were isolated from Scube1^{f/f} or Scube1^{f/f}; R26^{CreERT2} C57BL/6 mouse bone marrow, followed by transduction of MLL-AF9 retrovirus and three rounds of methylcellulose colony formation assay. After a third round of colony formation, the cells were intravenously injected into sub-lethally irradiated C57BL/6 mice. When the primary transplanted mice showed symptoms of disease, leukemic cells were isolated from bone marrow and secondary transplantation was performed. Two weeks after transplantation, established leukemia was confirmed by blast cells in peripheral blood. After disease establishment, five doses of tamoxifen were administered to inactivate Scube1. FIG. 5D shows a Kaplan-Meier curve showing survival of secondary transplanted mice. The median survival for Scube1^{f/f}−Tam (dashed blue line), Scube1^{f/f}+Tam (solid blue line), Scube1^{f/f}; R26^{CreERT2}−Tam (dashed red line), and Scube1^{f/f}; R26^{CreERT2}+Tam (solid red line) mice was 51.5, 51, 56, and 95 days respectively. FIG. 5E show spleen enlargement of secondary transplanted mice. Data are mean±SD from 3 independent experiments. **P<0.01.

FIGS. 6A-6E show that SCUBE1 binds FLT3 and promotes FLT3-LYN signaling. FIG. 6A shows a Venn diagram showing number of membrane proteins in the immediate vicinity of surface SCUBE1 identified by proteomic proximity labeling assay in THP-1 or NOMO-1 cells. Of 113 common proteins, 7 protein tyrosine kinases [symbol] 4 RTKs (FLT3, EPHB1, EPHB3, and INSR) and 3 TKs (LYN, JAK1, and TYK2) [symbol] were associated with or in proximity to SCUBE1. The antibody directed targeting of peroxidase (a combination of a primary mouse monoclonal anti-SCUBE1 antibody and an HRP-conjugated anti-mouse secondary antibody) to SCUBE1, followed by brief labeling with biotin-tyramide enabled proteins in the immediate vicinity of the target to be biotinylated. After cell lysis and capture by immobilized streptavidin, the biotinylated proteins were eluted with reducing agent and analyzed by liquid chromatography-mass spectrometry (LC-MS). The experiment was repeated with only a primary isotype control antibody for identifying nonspecific proteins. MS analysis confirmed that SCUBE1 protein was immunoprecipitated by the anti-SCUBE1 antibody under these conditions. FIG. 6B shows graphic diagrams showing the domain structure of SCUBE1 and deletion constructs of FLT3 used to map the interacting domain. FLAG epitope was added immediately after the signal peptide sequence at the NH₂-terminus of the SCUBE1 construct. Likewise, Myc epitope was tagged to the NH2-terminus of FLT3 full-length (FL) and its deletion mutants D1, D2, D3, D4, and D5. SP, signal peptide; Cys-Rich, cysteine-rich; TM, transmembrane domain; JM, juxtamembrane domain; The tyrosine kinase domain (TKD) is separated into two parts by a short region designated the kinase insert (KI). FIG. 6C shows molecular mapping of the interacting domains between SCUBE1 and FLT3. The expression plasmid encoding FLAG-tagged SCUBE1 was transfected alone or together with a series of Myc-tagged FLT3 constructs in HEK-293T cells for 2 days, then cell lysates underwent immunoprecipitation (IP), followed by western blot (WB) analysis with indicated antibodies to determine the protein-protein interactions. FIG. 6D shows phosphorylation of LYN analyzed with co-expression of FLT3 and/or SCUBE1 in HEK-293T cells. NH₂-terminus HIS-tagged LYN was transfected in HEK-293T cells alone or with FLAG-tagged SCUBE1 and/or Myc-tagged FLT3. Two days after transfection, cells were lysed and western blot analysis was performed. The activation of FLT3 was detected with anti-phospho-tyrosine (pY) antibody and total FLT3 activity was detected with anti-Myc antibody. The activation of LYN was detected with a specific anti-pLYN (Y397) antibody and total LYN activity was detected with anti-HIS antibody; SCUBE1 activity was detected with anti-FLAG antibody. Of note, because of its heavy N-linked glycosylation of the extracellular domain, FLT3 displays as two higher molecular masses on western blot analysis: one corresponds to 132 kDa as a not fully processed, partially glycosylated form and the other appears at 160 kDa, representing the mature, fully glycosylated FLT3. The quantified band intensities were normalized to loading controls and are mentioned below the corresponding bands. FIG. 6E shows the effect of SCUBE1 knockdown on phosphorylation/activation of FLT3-LYN-AKT signal cascade in THP-1 or NOMO-1 cells. To knock down SCUBE1, stable THP-1 or NOMO-1 cell lines carrying inducible SCUBE1-shRNA #1 or #2 were treated without (−) or with (+) doxycycline for 5 days. Western blot analysis was used to determine the phosphorylation status of FLT3 (pY), pLYN (Y397), pAKT (S473), or pERK1/2 (T202/Y204) or to quantify the corresponding total protein as controls. SCUBE1 was detected with anti-SCUBE1 #7 mAb. The quantified band intensities were normalized to loading controls and are mentioned below the corresponding bands.

FIGS. 7A and 7B shows internalization of the anti-SCUBE1 antibody. FIG. 7A shows internalization of the anti-SCUBE1 antibody overtime at 4° C. and 37° C. Images were acquired with a Zeiss LSM 510 confocal microscope, 40× (Zeiss), oil immersion lens, Zen software (Zeiss). Scale bar=20 NM. FIG. 7B shows localization of the anti-SCUBE1 antibody in THP-1 cells (SCUBE1 positive cells) and KG-1a cells (SCUBE1 negative cells) overtime. Human IgG 1 mg/ml was used for treatment 30 min before the addition of anti-SCUBE1 antibody to block FC to reduce nonspecific binding to FcγRs. To study localization, the anti-SCUBE1 antibody was detected using Alexa Fluor-488 (green), lysosomes were detected using LysoView 650 (red), and nucleus were detected using DAPI (blue). Yellow or orange fluorescence indicates co-localization of antibody to acidic lysosomal compartment. Images were acquired with a Zeiss LSM 700 confocal microscope, 63× (Zeiss), oil immersion lens, Zen software (Zeiss). Scale bar=5 μm.

FIGS. 8A-8D show production and in vitro characterization of an anti-SCUBE1 antibody-drug conjugate (ADC). FIG. 8A shows an exemplary anti-SCUBE1 ADC described herein. Tri-mannosyl anti-SCUBE1 antibody was conjugated to the anti-microtubule cytotoxic agent monomethyl auristatin E (MMAE) by a proteolytically cleavable DBCO-PEG3-VC-PAB linker with an average drug-to-antibody ratio of 4. DBCO, dibenzocyclooctyne; PEG, polyethylene glycol; VC, valine-citrulline; PAB, para aminobenzoate; GlcNAc: N-acetylglucosamine. FIG. 8B shows a reducing and non-reducing SDS-PAGE showing Coomassie blue staining of the parental anti-SCUBE1 (Si) antibody and an anti-Si ADC (anti-Sl-valine-citrulline [VC]-monomethyl auristatin [MMAE]) antibody on non-reducing and reducing SDS-PAGE. Of note, the non-reduced recombinant anti-Sl antibody was detected with molecular mass ˜180 kDa, whereas individual heavy or light chains are visible at ˜55 kDa or ˜25 kDa, respectively. FIG. 8C shows matrix-assisted laser desorption/ionization time-of-flight mass spectrometry demonstrating that the intact anti-SCUBE1 ADC was produced with an average drug-to-antibody ratio (DAR) of 3.89. The intact anti-S1-VC-4MMAE with 2 MMAEs on each arm of antibody has a peak at mass 156155.60 Da. “A” represents a molecular moiety of dibenzocylcooctyne (DBCO)-polyethylene glycol (PEG) 3-VC-para-aminobenzoate (PAB)-MMAE. “m” indicates uncertain modification of antibody. FIG. 8D shows that anti-SCUBE1 ADC induces cytotoxicity in AML cell lines. Assays were performed in the presence of the unconjugated anti-SCUBE1 antibody or anti-SCUBE1 ADC. MTT was used to measure cell viability after 5 days. The half maximal inhibitory concentration (IC50, nM) of anti-SCUBE1 ADC on killing the SCUBE1 expression in THP-1 or NOMO-1 cells is shown inside the graph. Note that the anti-SCUBE1 ADC did not exhibit antitumor efficacy on SCUBE1-negative KG-1a or K562 cells.

FIGS. 9A and 9B show a working model illustrating the mechanism of action of surface SCUBE1 in MLL-r leukemias and potential immunotherapy approach. FIG. 9A shows that SCUBE1 is a direct downstream target of transcriptional regulatory complex of HOXA9/MEIS1, which are upregulated by MLL-fusion proteins such as MLL-AF9 and are essential for maintaining leukemic transformation. Surface SCUBE1 plays a critical pathogenic function in MLL-r leukemias by acting as a FLT3 coreceptor via its spacer region and the COOH-terminal CUB domain in facilitating the interaction between FLT3 ligand (FLT3L) and FLT3, augmenting downstream LYN-AKT activation (tyrosine phosphorylation) for leukemic cell proliferation and survival, thus promoting leukemogenesis. FIG. 9B shows that surface expression of SCUBE1 on MLL-r leukemia provides the opportunity for its potential use as a target for immunotherapy because an anti-SCUBE1 ADC conjugated to an antimitotic agent MMAE leads to significant cell killing specifically on MLL-AF9 leukemias.

FIGS. 10A-10C show that the anti-SCUBE1 ADC reduced tumor growth of THP-1 cells in a subcutaneous model. FIG. 10A shows the anti-SCUBE1 ADC-reduced tumor volume of THP-1 cells. Data were evaluated by multiple-sample Student t test (n=6). FIG. 10B shows tumors (upper panel) collected from sacrificed mice and their weight (lower panel). Data were evaluated with Mann-Whitney test. Scale bar=1 cm. FIG. 10C shows body weight change after transplantation until the completion of the experiment. Data are mean±SD from 3 independent experiments. **P<0.01.

FIG. 11A shows a schematic representation of the treatment procedure in an orthotopic model. FIG. 11B shows representative images of bioluminescent imaging (BLI) on different days after the IgG and anti-SCUBE1 ADC treatment. FIG. 11C shows quantitative data of total flux measured from the images in FIG. 11B. FIG. 11D shows overall survival curves of the IgG-treated mice and anti-SCUBE1 ADC-treated mice. N=5, **P<0.001.

### DETAILED DESCRIPTION OF THE INVENTION

It must be noted that, as used in this specification and the appended claims, the singular forms “a,” “an,” and “the” include plural referents unless the content clearly dictates otherwise.

As used herein, the term “complementarity determining region” (CDR) refers to a hypervariable region found both in the light chain and the heavy chain variable domains of an antibody. The more highly conserved portions of variable domains are referred to as framework regions (FRs). The amino acid positions that delineate a hypervariable region of an antibody can vary, depending on the context and the various definitions known in the art. Some positions within a variable domain may be viewed as hybrid hypervariable positions in that these positions can be deemed to be within a hypervariable region under one set of criteria while being deemed to be outside a hypervariable region under a different set of criteria. One or more of these positions can also be found in extended hypervariable regions. The antibodies described herein may contain modifications in these hybrid hypervariable positions. The CDRs in each chain are held together in close proximity by the framework regions in the order FR1-CDR1-FR2-CDR2-FR3-CDR3-FR4 and, with the CDRs from the other antibody chains, contribute to the formation of the target binding site of antibodies (see Kabat et al., Sequences of Proteins of Immunological Interest, National Institute of Health, Bethesda, Md., 1987). Numbering of immunoglobulin amino acid residues is performed according to the immunoglobulin amino acid residue numbering system of Kabat et al., IMGT, Chothia or other system known in the art.

As used herein, the term “antibody” refers to an immunoglobulin molecule that specifically binds to, or is immunologically reactive with, a particular antigen. An antibody includes, but is not limited to, monoclonal antibodies, polyclonal antibodies, multispecific antibodies (e.g., bispecific antibodies), genetically engineered, and otherwise modified forms of antibodies, including but not limited to de-immunized antibodies, chimeric antibodies, humanized antibodies, heteroconjugate antibodies (e.g., bi- tri- and quad-specific antibodies, diabodies, triabodies, and tetrabodies), and antibody fragments (i.e., antigen binding fragments of antibodies), including, for example, Fab′, F(ab′)₂, Fab, Fv, rlgG, and scFv fragments, so long as they exhibit the desired antigen-binding activity.

The term “antigen-binding fragment,” as used herein, refers to one or more portions of an antibody that retain the ability to specifically bind to a target antigen. The antigen-binding function of an antibody can be performed by fragments of a full-length antibody. The antibody fragments can be, for example, a Fab, F(ab)₂, scFv, diabody, a triabody, an affibody, a nanobody, an aptamer, or a domain antibody. Examples of binding fragments encompassed of the term “antigen-binding fragment” of an antibody include, but are not limited to: (i) a Fab fragment, a monovalent fragment consisting of the V_{L}, V_{H}, C_{L}, and C_{H}1 domains; (ii) a F(ab)₂ fragment, a bivalent fragment containing two Fab fragments linked by a disulfide bridge at the hinge region; (iii) a Fd fragment consisting of the V_{L}, V_{H}, C_{L}, and C_{H}1 domains; (iv) a Fv fragment consisting of the V_{L} and V_{H} domains of a single arm of an antibody, (v) a dAb including V_{L} and V_{H} domains; (vi) a dAb fragment that consists of a V_{H} domain; (vii) a dAb which consists of a V_{H} or a V_{L} domain; (viii) an isolated complementarity determining region (CDR); and (ix) a combination of two or more (e.g., two, three, four, five, or six) isolated CDRs which may optionally be joined by a synthetic linker. Furthermore, although the two domains of the Fv fragment, V_{L} and V_{H}, are coded for by separate genes, they can be joined, using recombinant methods, by a linker that enables them to be made as a single protein chain in which the V_{L} and V_{H} regions pair to form monovalent molecules. These antibody fragments can be obtained using conventional techniques known to those of skill in the art, and the fragments can be screened for utility in the same manner as intact antibodies. Antigen-binding fragments can be produced by recombinant DNA techniques, enzymatic or chemical cleavage of intact immunoglobulins, or, in certain cases, by chemical peptide synthesis procedures known in the art.

As used herein, the term “conjugate” or “antibody drug conjugate” or “ADC” refers to a substance made up of a monoclonal antibody chemically linked to a drug. The monoclonal antibody binds to specific proteins or receptors found on certain types of cells, including cancer cells. The linked drug enters these cells and kills them without harming other cells. An ADC is formed by the chemical bonding of a reactive functional group of one molecule, such as an antibody or antigen-binding fragment thereof, with an appropriately reactive functional group of another molecule, such as a cytotoxin described herein. Conjugates may include a linker between the two molecules bound to one another, e.g., between an antibody and a cytotoxin. Examples of linkers that can be used for the formation of a conjugate include peptide-containing linkers, such as those that contain naturally occurring or non-naturally occurring amino acids, such as D-amino acids. Linkers can be prepared using a variety of strategies described herein and known in the art.

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.”

The terms “antibody-dependent cell-mediated cytotoxicity” or “ADCC” refer to a form of cytotoxicity in which a polypeptide comprising an Fc domain, e.g., an antibody, bound onto Fc receptors (FcRs) present on certain cytotoxic cells (e.g., primarily NK cells, neutrophils, and macrophages) and enables these cytotoxic effector cells to bind specifically to an antigen-bearing “target cell” and subsequently kill the target cell with cytotoxins. It is contemplated that, in addition to antibodies and fragments thereof, other polypeptides comprising Fc domains, e.g., Fc fusion proteins and Fc conjugate proteins, having the capacity to bind specifically to an antigen-bearing target cell will be able to effect cell-mediated cytotoxicity.

As used herein, the terms “treat,” “treatment,” and “treating” refer to an approach for obtaining beneficial or desired results, for example, clinical results. For the purposes of this disclosure, beneficial or desired results may include inhibiting or suppressing the initiation or progression of a disease; ameliorating, or reducing the development of, symptoms of a disease; or a combination thereof.

“Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared. For purposes herein, however, % amino acid sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, California, or may be compiled from the source code. The ALIGN-2 program should be compiled for use on a UNIX operating system, including digital UNIX V4.0D. All sequence comparison parameters are set by the ALIGN-2 program and do not vary. In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows:

100 times the fraction X/Y

where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

As used herein, the terms “preventing” and “prevention” are used interchangeably with “prophylaxis” and can mean complete prevention of an infection, or prevention of the development of symptoms of a disease; a delay in the onset of a disease or its symptoms; or a decrease in the severity of a subsequently developed disease or its symptoms.

As used herein an “effective amount” refers to an amount of an antibody or ADC sufficient to reduce at least one symptom of a disease.

As interchangeably used herein, the terms “individual,” “subject,” “host,” and “patient,” refer to a mammal, including, but not limited to, murines (rats, mice), non-human primates, humans, canines, felines, ungulates (e.g., equines, bovines, ovines, porcines, caprines), etc. Particularly, the subject is vaccinated.

As used herein, the term “pharmaceutically acceptable” means being approved by a regulatory agency of a U.S. Federal or a state government or listed in the U.S. Pharmacopeia, European Pharmacopeia or other generally recognized pharmacopeia for use in mammals, and more particularly in humans. These compositions can be useful as a vaccine and/or antigenic compositions for inducing a protective immune response in a vertebrate.

The present invention relates to an anti-SCUBE1 antibody exhibiting a high internalization capacity and an anti-SCUBE1 antibody-drug conjugate for killing SCUBE1-expressing cancer cells. Particularly, the present disclosure demonstrates by using a conditional knockout mouse model that Scube1 is required for both the initiation and maintenance of MLL-AF9-induced leukemogenesis in vivo. Further proteomic, molecular and biochemical analyses reveal that the membrane-tethered SCUBE1 binds to the FLT3 ligand and the extracellular ligand-binding domains of FLT3, thus facilitating activation of the signal axis FLT3-LYN (a nonreceptor TK) to initiate leukemic growth and survival signals.

Rearrangement of the mixed lineage leukemia gene (MLL; also known as lysine methyltransferase 2A, KMT2A) on chromosomal band 11q23 accounts for 10% of all human leukemias and manifests as acute lymphoblastic leukemia (ALL) or acute myeloid leukemia (AML). Although conventional chemotherapy has been improved for leukemia, patients with MLL-rearranged (MLL-r) leukemia generally exhibit relatively poor treatment response and poor prognosis. Gene expression of SCUBE1 is highly upregulated in MLL-r leukemia. In addition, zebrafish Scube1 is implicated in primitive hematopoiesis by modulating BMP signal activity during embryogenesis. However, whether SCUBE1 is actively involved in the initiation and maintenance of MLL-r leukemogenesis and if so, whether SCUBE1 represents a potential target to treat MLL-r leukemia remain largely unknown.

The present disclosure first shows that SCUBE1 is cooperatively upregulated by homeobox A9 (HOXA9) and Meis homeobox 1 (MEIS1) in MLL-r leukemia. Molecular, genetic, proteomic and biochemical studies further demonstrate that the membrane-tethered SCUBE1 is essential for the initiation and maintenance of MLL-r leukemias by augmenting the proliferative and survival signaling axis mediated by Fms-like receptor tyrosine kinase 3 (FLT3)-Lck/Yes-related novel protein tyrosine kinase (LYN). In addition, present disclosure demonstrates that an anti-SCUBE1 monomethyl auristatin E (an anti-microtubule cytotoxin) antibody-drug conjugate (ADC) shows specific and enhanced anti-leukemic effects in SCUBE1-positive MLL-r AML cells. These results suggest that targeting the cell-surface SCUBE1 might be an efficient and promising strategy for treating MLL-r AML.

### Anti-SCUBE1 Antibodies

The present disclosure is also based in part on the discovery that antibodies, or antigen-binding fragments thereof, capable of binding SCUBE1 can be used as therapeutic agents alone or as ADCs to treat SCUBE1-expressing cancer. These therapeutic activities can be caused, for instance, by the binding of anti-SCUBE1 antibodies, or antigen-binding fragments thereof, to SCUBE1 expressed on the surface of a cell and subsequently inducing cell death.

Antibodies and antigen-binding fragments capable of binding SCUBE1 described herein comprise the following heavy chain variable region (VH) sequence and light chain variable region (VL) sequence (wherein the amino acid sequences marked in bold are the CDRs).

VH of the M20031101 (SEQ ID NO: 7):
EVQLQQSGPELVKPGASVKMSCKASGYTFTSYAMHWVKQKPGQGLEWIGYINPYND
VSRYNEKFQGKATLTSDKSSNTAYMELSSLTSEDSAVYYCEARPTSAPYFDVFGTGTT
VTVSS
VL of the M20031101 (SEQ ID NO: 8)
DIVMSQSPSSLAASVGEKVTMTCKSSQSLLNSRTRKNYLAWYQQKPGQSPKLLIYWTS
TRESGVPDRFTGSGSGTDFTLTISSVQTEDLAIYYCKQSYNLFTFGSGTKLEIKR
The nucleotide sequences of the VH and the VL are listed below.
Nucleotide sequences of the VH (SEQ ID NO: 9)
(SEQ ID NO: 9)
GAGGTCCAGCTGCAGCAGTCTGGACCTGAACTGGTAAAGCCTGGGGCTTCAGTGAAGATG
TCCTGCAAGGCTTCTGGATACACATTCACTAGCTATGCTATGCACTGGGTGAAGCAGAAGC
CTGGGCAGGGCCTTGAGTGGATTGGATATATTAATCCTTACAATGATGTTAGTAGGTACAAT
GAGAAGTTCCAAGGCAAGGCCACACTGACTTCAGACAAATCCTCCAACACAGCCTACATGG
AGCTCAGCAGCCTGACCTCTGAGGACTCTGCGGTCTATTACTGTGAGGCCCGCCCTACTTC
GGCTCCGTACTTCGATGTCTTTGGCACAGGGACCACGGTCACCGTCTCCTCA
Nnucleotide sequences of the VL (SEQ ID NO: 10)
GACATTGTGATGTCACAGTCTCCATCCTCCCTGGCTGCGTCAGTGGGAGAGAAGGTCACTA
TGACCTGCAAATCCAGTCAGAGTCTGCTCAACAGTAGAACCCGAAAGAACTACTTGGCTTG
GTATCAGCAGAAACCAGGGCAGTCTCCTAAACTGCTGATCTACTGGACATCCACTAGGGAA
TCTGGGGTCCCTGATCGCTTCACAGGCAGTGGATCTGGGACAGATTTCACTCTCACCATCA
GCAGTGTGCAGACTGAAGACCTGGCAATTTATTACTGCAAGCAATCTTATAATCTATTCACG
TTCGGCTCGGGGACAAAGTTGGAAATAAAACGG

The sequences of the VH and VL and their nucleotide sequences are listed below (wherein the sequences marked in bold are the CDRs).

```
VH
  1 2 3 4 5 6 7 8 9 10 11 12 13 14 15 16
  E V Q L Q Q S G P E L V K P G A
GAG GTC CAG CTG CAG CAG TCT GGA CCT GAA CTG GTA AAG CCT GGG GCT
  17 18 19 20 21 22 23 24 25 26 27 28 29 30 31 32
  S V K M S C K A S G Y T F T S Y
TCA GTG AAG ATG TCC TGC AAG GCT TCT GGA TAC ACA TTC ACT AGC TAT
  33 34 35 36 37 38 39 40 41 42 43 44 45 46 47 48
  A M H W V K Q K P G Q G L E W I
GCT ATG CAC TGG GTG AAG CAG AAG CCT GGG CAG GGC CTT GAG TGG ATT
  49 50 51 52 53 54 55 56 57 58 59 60 61 62 63 64
  G Y I N P Y N D V S R Y N E K F
GGA TAT ATT AAT CCT TAC AAT GAT GTT AGT AGG TAC AAT GAG AAG TTC
  65 66 67 68 69 70 71 72 73 74 75 76 77 78 79 80
  Q G K A T L T S D K S S N T A Y
CAA GGC AAG GCC ACA CTG ACT TCA GAC AAA TCC TCC AAC ACA GCC TAC
  81 82 83 84 85 86 87 88 89 90 91 92 93 94 95 96
  M E L S S L T S E D S A V Y Y C
ATG GAG CTC AGC AGC CTG ACC TCT GAG GAC TCT GCG GTC TAT TAC TGT
  97 98 99 100 101 102 103 104 105 106 107 108 109 110 111 112
  E A R P T S A P Y F D V R G T G
GAG GCC CGC CCT ACT TCG GCT CCG TAC TTC GAT GTC TTT GGC ACA GGG
  113 114 115 116 117 118 119
  T T V T V S S
ACC ACG GTC ACC GTC TCC TCA
  1 2 3 4 5 6 7 8 9 10 11 12 13 14 15 16
  D I V M S Q S P S S L A A S V G
GAC ATT GTG ATG TCA CAG TCT CCA TCC TCC CTG GCT GCG TCA GTG GGA
  17 18 19 20 21 22 23 24 25 26 27 28 29 30 31 32
  E K V T M T C K S S Q S L L N S
GAG AAG GTC ACT ATG ACC TGC AAA TCC AGT CAG AGT CTG CTC AAC AGT
  33 34 35 36 37 38 39 40 41 42 43 44 45 46 47 48
  R T R K N Y L A W Y Q Q K P G Q
AGA ACC CGA AAG AAC TAC TTG GCT TGG TAT CAG CAG AAA CCA GGG CAG
  49 50 51 52 53 54 55 56 57 58 59 60 61 62 63 64
  S P K L L I Y W T S T R E S G V
TCT CCT AAA CTG CTG ATC TAC TGG ACA TCC ACT AGG GAA TCT GGG GTC
  65 66 67 68 69 70 71 72 73 74 75 76 77 78 79 80
  P D R F T G S G S G T D F T L T
CCT GAT CGC TTC ACA GGC AGT GGA TCT GGG ACA GAT TTC ACT CTC ACC
  81 82 83 84 85 86 87 88 89 90 91 92 93 94 95 96
  I S S V Q T E D L A I Y Y C K Q
ATC AGC AGT GTG CAG ACT GAA GAC CTG GCA ATT TAT TAC TGC AAG CAA
  97 98 99 100 101 102 103 104 105 106 107 108 109 110 111 112
  S Y N L F T F G S G T K L E I K
TCT TAT AAT CTA TTC ACG TTC GGC TCG GGG ACA AAG TTG GAA ATA AAA
```

The antibody according to the disclosure can be full-length or may comprise only an antigen-binding portion (for example, a Fab, F(ab′)₂ or scFv fragment), and may be modified to affect functionality as needed.

Various techniques known to persons of ordinary skill in the art can be used to determine whether an antibody “specifically binds to one or more amino acids” within a polypeptide or protein. Exemplary techniques include, e.g., routine cross-blocking assay such as that described Antibodies, Harlow and Lane (Cold Spring Harbor Press, Cold Spring Harb., N.Y.), alanine scanning mutational analysis, peptide blots analysis (Reineke, 2004, Methods Mol Biol 248:443-463), and peptide cleavage analysis. In addition, methods such as epitope excision, epitope extraction and chemical modification of antigens can be employed (Tomer, 2000, Protein Science 9:487-496). Another method that can be used to identify the amino acids within a polypeptide with which an antibody specifically binds is hydrogen/deuterium exchange detected by mass spectrometry. In general terms, the hydrogen/deuterium exchange method involves deuterium-labeling the protein of interest, followed by binding the antibody to the deuterium-labeled protein. Next, the protein/antibody complex is transferred to water to allow hydrogen-deuterium exchange to occur at all residues except for the residues protected by the antibody (which remain deuterium-labeled). After dissociation of the antibody, the target protein is subjected to protease cleavage and mass spectrometry analysis, thereby revealing the deuterium-labeled residues which correspond to the specific amino acids with which the antibody interacts. See, e.g., Ehring (1999) Analytical Biochemistry 267(2):252-259; Engen and Smith (2001) Anal. Chem. 73:256A-265A.

The antibody also includes an antigen-binding fragment of a full antibody molecule. An antigen-binding fragment of an antibody may be derived, e.g., from full antibody molecules using any suitable standard techniques such as proteolytic digestion or recombinant genetic engineering techniques involving the manipulation and expression of DNA encoding antibody variable and optionally constant domains. Such DNA is known and/or is readily available from, e.g., commercial sources, DNA libraries (including, e.g., phage-antibody libraries), or can be synthesized. The DNA may be sequenced and manipulated chemically or by using molecular biology techniques, for example, to arrange one or more variable and/or constant domains into a suitable configuration, or to introduce codons, create cysteine residues, modify, add or delete amino acids, etc.

Non-limiting examples of an antigen-binding fragment include: (i) Fab fragments; (ii) F(ab′)₂ fragments; (iii) Fd fragments; (iv) Fv fragments; (v) single-chain Fv (scFv) molecules; (vi) dAb fragments; and (vii) minimal recognition units consisting of the amino acid residues that mimic the hypervariable region of an antibody (e.g., an isolated complementarity determining region (CDR) such as a CDR3 peptide), or a constrained FR3-CDR3-FR4 peptide. Other engineered molecules, such as domain-specific antibodies, single domain antibodies, domain-deleted antibodies, chimeric antibodies, CDR-grafted antibodies, diabodies, triabodies, tetrabodies, minibodies, nanobodies (e.g. monovalent nanobodies, bivalent nanobodies, etc.), small modular immunopharmaceuticals (SMIPs), and shark variable IgNAR domains, are also encompassed by the expression “antigen-binding fragment,” as used herein.

An antigen-binding fragment of an antibody typically comprises at least one variable domain. The variable domain may be of any size or amino acid composition and will generally comprise at least one CDR which is adjacent to or in frame with one or more framework sequences. In antigen-binding fragments having a V_{H} domain associated with a V_{L} domain, the V_{H} and V_{L} domains may be situated relative to one another in any suitable arrangement. For example, the variable region may be dimeric and contain V_{H}-V_{H}, V_{H}-V_{L} or V_{L}-V_{L} dimers. Alternatively, the antigen-binding fragment of an antibody may contain a monomeric V_{H} or V_{L} domain.

Antibodies may be produced using recombinant methods and compositions known in the art. In one embodiment, isolated nucleic acid encoding an anti-SCUBE1 antibody described herein is provided. Such nucleic acid may encode an amino acid sequence comprising the VL and/or an amino acid sequence comprising the VH of the antibody (e.g., the light and/or heavy chains of the antibody). In a further embodiment, one or more vectors (e.g., expression vectors) comprising such nucleic acid are provided.

For recombinant production of an anti-SCUBE1 antibody, nucleic acid encoding an antibody, e.g., as described above, is isolated and inserted into one or more vectors for further cloning and/or expression in a host cell. Such nucleic acid may be readily isolated and sequenced using conventional procedures (e.g., by using oligonucleotide probes that are capable of binding specifically to genes encoding the heavy and light chains of the antibody). Suitable host cells for cloning or expression of antibody-encoding vectors include prokaryotic or eukaryotic cells described herein.

In one embodiment, the anti-SCUBE1 antibody, or antigen binding fragment thereof, comprises variable regions having an amino acid sequence that is at least about 80%, about 85%, about 90%, about 91%, about 92%, about 93%, about 94%, about 95%, about 96%, about 97%, about 98%, about 99% or more identical to the SEQ ID Nos disclosed herein. Alternatively, the anti-SCUBE1 antibody, or antigen binding fragment thereof, comprises CDRs comprising the SEQ ID Nos disclosed herein with framework regions of the variable regions described herein having an amino acid sequence that is at least about 80%, about 85%, about 90%, about 91%, about 92%, about 93%, about 94%, about 95%, about 96%, about 97%, about 98%, about 99% or more identical to the SEQ ID NOs disclosed herein.

In one embodiment, the anti-SCUBE1 antibody, or antigen binding fragment thereof, comprises a heavy chain variable region and a heavy chain constant region having an amino acid sequence that is disclosed herein. In another embodiment, the anti-SCUBE1 antibody, or antigen binding fragment thereof, comprises a light chain variable region and a light chain constant region having an amino acid sequence that is disclosed herein. In yet another embodiment, the anti-SCUBE1 antibody, or antigen binding fragment thereof, comprises a heavy chain variable region, a light chain variable region, a heavy chain constant region and a light chain constant region having an amino acid sequence that is disclosed herein.

### Antibody Drug Conjugates (ADCs)

Anti-SCUBE1 antibodies, and antigen-binding fragments thereof, described herein can be conjugated (linked) to a cytotoxin. In particular, the anti-SCUBE1 ADCs include an antibody (or an antigen-binding fragment thereof) conjugated (i.e., covalently attached by a linker) to a cytotoxic moiety (or cytotoxin). In various embodiments, the cytotoxic moiety exhibits reduced or no cytotoxicity when bound in a conjugate, but resumes cytotoxicity after cleavage from the linker. In various embodiments, the cytotoxic moiety maintains cytotoxicity without cleavage from the linker.

Antibodies, and antigen-binding fragments thereof, described herein (e.g., antibodies, and antigen-binding fragments thereof, that recognize and bind SCUBE1) can be conjugated (or linked) to a cytotoxin.

ADCs of the present disclosure therefore may be of the general formula Ab-(Z-L-D)ₙ wherein an antibody or antigen-binding fragment thereof (Ab) is conjugated (covalently linked) to linker (L), through a chemical moiety (Z), to a cytotoxic moiety (“drug,” D). “n” represents the number of drugs linked to the antibody.

Accordingly, the antibody or antigen-binding fragment thereof may be conjugated to a number of drug moieties as indicated by integer n, which represents the average number of cytotoxins per antibody, which may range, e.g., from about 1 to about 20. The average number of drug moieties per antibody in preparations of ADC from conjugation reactions may be characterized by conventional means such as mass spectroscopy, ELISA assay, and HPLC. The quantitative distribution of ADC in terms of n may also be determined. In some instances, separation, purification, and characterization of homogeneous ADC where n is a certain value from ADC with other drug loadings may be achieved by means such as reverse phase HPLC or electrophoresis.

Cytotoxins suitable for use with the compositions and methods described herein include DNA-intercalating agents, (e.g., anthracyclines), agents capable of disrupting the mitotic spindle apparatus (e.g., Vinca alkaloids, maytansine, maytansinoids, and derivatives thereof), RNA polymerase inhibitors (e.g., an amatoxin, such as .alpha.-amanitin, and derivatives thereof), and agents capable of disrupting protein biosynthesis (e.g., agents that exhibit rRNA N-glycosidase activity, such as saporin and ricin A-chain), among others known in the art.

In some embodiments, the cytotoxin is a microtubule-binding agent (for instance, maytansine or a maytansinoid), an amatoxin, Pseudomonas exotoxin A, deBouganin, diphtheria toxin, saporin, an auristatin, an anthracycline, a calicheamicin, irinotecan, SN-38, a duocarmycin, a pyrrolobenzodiazepine, a pyrrolobenzodiazepine dimer, an indolinobenzodiazepine, an indolinobenzodiazepine dimer, or a variant thereof, or another cytotoxic compound described herein or known in the art. Antibodies and antigen-binding fragments thereof described herein can be conjugated to a cytotoxin that is a maytansinoid. In some embodiments, the microtubule binding agent is a maytansine, a maytansinoid or a maytansinoid analog. Maytansinoids are mitototic inhibitors which bind microtubules and act by inhibiting tubulin polymerization. Examples of suitable maytansinoids include esters of maytansinol, synthetic maytansinol, and maytansinol analogs and derivatives; for example, Monomethyl auristatin E (MMAE).

The linker used herein has a functionality that is capable of connecting the cytotoxin (D) and the chemical moiety (Z). In some embodiments, the linker (L), to which the antibody or antigen-binding fragment thereof described herein is conjugated, may be a cleavable linker. In one embodiment, the cleavable linker is a proteolytically cleavable linker such as a peptidase labile linker or a esterase labile linker. In one embodiment, the proteolytically cleavable linker comprises a valine-citrulline moiety. In one further embodiment, the proteolytically cleavable linker is a DBCO-PEG3-VC-PAB linker (DBCO, dibenzocyclooctyne; PEG, polyethylene glycol; VC, valine-citrulline; PAB, para aminobenzoate).

In some embodiments, the chemical moiety (Z), through which the antibody or antigen-binding fragment thereof described herein is conjugated to the linker (L), may be an oligosaccharide moiety. For example, the oligosaccharide moiety is represented by the following formula:

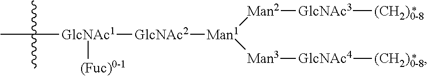

wherein * denotes the end to which the linker (L) is connected.

### Administration Methods

ADCs, antibodies, or antigen-binding fragments thereof, as described herein can be administered to a patient (e.g., a human patient suffering from cancer, an autoimmune disease, or in need of hematopoietic stem cell transplant therapy) in a variety of dosage forms.

The anti-SCUBE1 ADCs, antibodies, or antigen-binding fragments, described herein may be administered by a variety of routes, such as orally, transdermally, subcutaneously, intranasally, intravenously, intramuscularly, intraocularly, or parenterally. The most suitable route for administration in any given case will depend on the particular antibody, or antigen-binding fragment, administered, the patient, pharmaceutical formulation methods, administration methods, the patient's age, body weight, sex, severity of the diseases being treated, the patient's diet, and the patient's excretion rate.

The disclosure provides pharmaceutical compositions comprising the antibody or antigen-binding fragment thereof. The pharmaceutical compositions of the disclosure are formulated with suitable diluents, carriers, excipients, and other agents that provide improved transfer, delivery, tolerance, and the like. The compositions may be formulated for specific uses, such as for veterinary uses or pharmaceutical uses in humans. The form of the composition and the excipients, diluents and/or carriers used will depend upon the intended uses of the antibody and, for therapeutic uses, the mode of administration. A multitude of appropriate formulations can be found in the formulary known to all pharmaceutical chemists: Remington's Pharmaceutical Sciences, Mack Publishing Company, Easton, Pa. These formulations include, for example, powders, pastes, ointments, jellies, waxes, oils, lipids, lipid (cationic or anionic) containing vesicles (such as LIPOFECTIN™, Life Technologies, Carlsbad, Calif.), DNA conjugates, anhydrous absorption pastes, oil-in-water and water-in-oil emulsions, emulsions carbowax (polyethylene glycols of various molecular weights), semi-solid gels, and semi-solid mixtures containing carbowax. See also Powell et al. “Compendium of excipients for parenteral formulations” PDA (1998) J Pharm Sci Technol 52:238-311.

### EXAMPLE

The following examples are put forth so as to provide those of ordinary skill in the art with a description of how the compositions and methods described herein may be used, made, and evaluated, and are intended to be purely exemplary of the present disclosure and are not intended to limit the scope of what the inventors regard as their invention.

### Methods

### Patients

The microarray data of patient samples were derived from a previous report.18 The study was approved by Research Ethics Committee of National Taiwan University Hospital, Taiwan.

### Mice

Our investigation conformed to the Guide for the Care and Use of Laboratory Animals published by the US National Institutes of Health (NIH Publication No. 85-23, revised 1996). All experimental procedures were performed according to the protocol approved by the Institutional Animal Care and Utilization Committee, Academia Sinica (Protocol 20-12-1922). NSG (NOD/SCID/IL-2Rγc−/−) mice were bred in-house at the Institute of Biomedical Sciences, Academia Sinica, Taiwan, animal facility.

### Chromatin Immunoprecipitation (ChIP)

The ChIP assay was performed as described (Prange K H M, Mandoli A, Kuznetsova T, et al. MLL-AF9 and MLL-AF4 oncofusion proteins bind a distinct enhancer repertoire and target the RUNX1 program in 11q23 acute myeloid leukemia. Oncogene. 2017; 36(23):3346-3356) with modification.

### In Vivo Leukemogenesis Study

NOD/SCID/IL-2Rγc˜/˜ (NSG) mice 8 to 10 weeks old were sub-lethally irradiated and injected intravenously (IV) with leukemia cells (THP1 or NOMO-1 with inducible shRNA #2). After injection, mice were randomly distributed and treated or not with doxycycline in drinking water. The animals were sacrificed with signs of distress.

### Methylcellulose Colony-Formation Assay and Bone-Marrow Transplantation

Methylcellulose colony-formation assay was performed as described 20, with modification. Briefly, cKit positive bone-marrow hematopoietic cells from 8- to 10-week-old Scube1 KO, WT, Scube1^{f/f}, or Scube1^{f/f}; R26^{CreERT2} C57BL/6 mice were transduced with MLL-AF9 retrovirus and/or SCUBE1 lentivirus isolated from HEK293T cells. Then cells were cultured in methylcellulose media supplemented with SCF, IL-3 and IL-6, and GM-CSF. After three rounds of re-plating, cells were primarily and secondarily transplanted into syngeneic mice.

### Proximity Ligation Assay

THP-1 or NOMO-1 cells were incubated with anti-SCUBE1 or an isotype control primary antibody followed by HRP-conjugated secondary antibody. Then biotin-tyramide along with H₂O₂ was briefly added to biotinylate the SCUBE1 proximal proteins. The biotinylated proteins were analyzed by liquid chromatography-mass spectrometry (LC-MS) after cell lysis.

### Internalization Assay

Anti-SCUBE1 antibody was labelled with Alexa Fluor 488 by using the commercial Antibody Labeling Kit (Thermo Fisher Scientific) according to the manufacturer's protocol. An amount of 1 mg/mL human IgG was added to cells to reduce nonspecific binding to FcgRs at 1 h before adding anti-SCUBE1 antibody. Anti-SCUBE1 antibody was added to cells for different times as indicated at 10 μg/ml. For lysosomal trafficking, cells were first incubated with LysoView 650 (Biotium) for 2 h under growth conditions to label the acidic compartments. Images were acquired at different times by using an LSM700 confocal microscope.

Cell Viability Assay with Antibody-Drug Conjugate (ADC)

Leukemic cells were incubated with serial dilutions of anti-SCUBE1 or anti-SCUBE1-VC-MMAE. Cells were incubated in normal culture condition for 5 days, followed by MTT [3-(4,5-dimethylthiazol-2-yl)-2,5-diphenyltetrazolium bromide] assay.

### Results

### Example 1—SCUBE1 is Highly Expressed in MLL-r AML and Predicts Poor Prognosis in De Novo AML

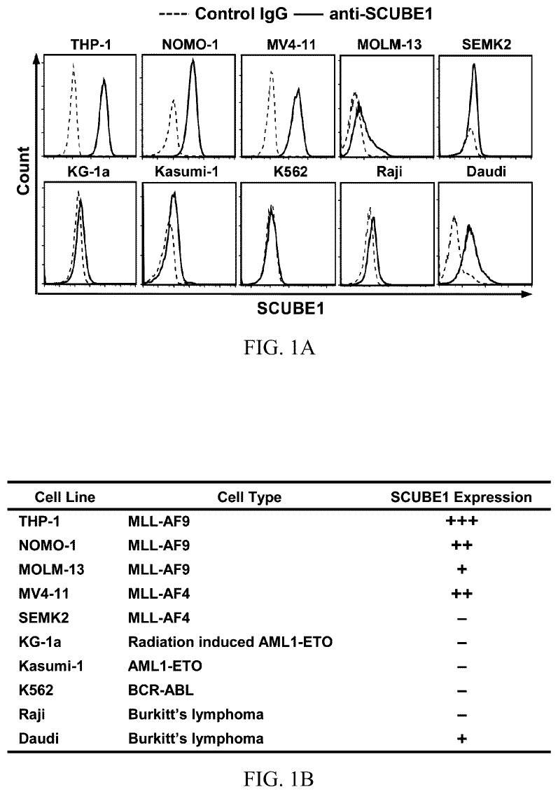

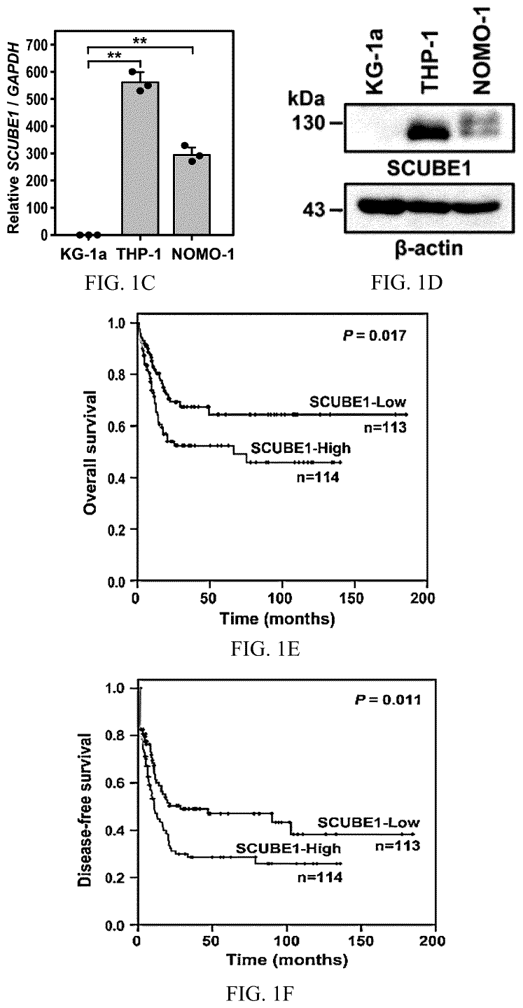

Previous transcriptomic profiling independently and reproducibly identified SCUBE1 as a highly overexpressed gene in MLL-r AML, including in one of the most common MLL translocations t (9;11) (p22; q23) resulting in MLL fused to AF9 (MLL-AF9). However, whether SCUBE1 is expressed at the protein level and whether its expression level has any prognostic value in AML remains unclear. We verified that along with mRNA expression, SCUBE1 is highly expressed on the cell surface of two MLL-AF9 AML cell lines (THP-1 and NOMO-1) but not in a non-MLL-r AML cell line, KG-1a [prone to formation of the t(8:21)(q22;q22)-associated AML1-ETO fusion gene], as determined by western blot analysis or flow cytometry analysis with a previously generated anti-SCUBE1 monoclonal antibody (Liao W J, Wu M Y, Peng C C, Tung Y C, Yang R B. Epidermal growth factor-like rep eats of SCUBE1 derived from platelets are critical for thrombus formation. Cardiovasc Res. 2020; 116(1):193-201) (FIGS. 1A-D). Of note, SCUBE1 is also highly expressed in a broader spectrum of hematological malignancies including MLL-AF4 (MV4-11) leukemic cells as well as Burkitt's lymphoma (Daudi) cells (FIGS. 1A and B). In addition, after surveying the public database at the cBioPortal for Cancer Genomics (cbioportal.org), we did not identify genomic gain or amplification nor activated mutation of SCUBE1 gene in AML or myelodysplastic syndromes (MDS) cohorts). These data suggest that SCUBE1 upregulation might occur in AML cells at the transcriptional rather than genomic level.

We further interrogated a previously published gene expression profiling dataset of bone-marrow mononuclear cells from 227 de novo AML patients (Okamoto M, Hayakawa F, Miyata Y, et al. Lyn is an important component of the signal transduction pathway specific to FLT3/ITD and can be a therapeutic target in the treatment of AML with FLT3/ITD. Leukemia. 2007; 21(3):403-10). High SCUBE1 expression is associated with a high white blood cell (WBC) count (P<0.001) or high blast cell count (P<0.001). Patients with M4 or M5 monoblastic subtypes according to the French-American-British (FAB) classification frequently have high SCUBE1 expression (P<0.001 and P<0.001, respectively). In line with previous expression profiling studies, high SCUBE1 expression is significantly associated with MLL abnormalities including MLL-r/MLL-partial tandem duplication (MLL-PTD). In addition, overall survival is shorter (median 66.1 months vs not reached; log-rank P=0.017), as was disease-free survival (median 9.4 vs 27.0 months; log-rank P=0.011) with high as compared to low SCUBE1 expression after a median follow up of 57.0 months (FIGS. 1E and F). On multivariate analysis, besides age or WBC count, we also used 2017 European LeukemiaNet (ELN) risk stratification for analysis including more comprehensive poor prognostic genetic factors and observed that high SCUBE1 expression remains an independent prognostic factor for overall survival (hazard ratio 1.663, 95% confidence interval 1.026-2.696). Our results demonstrate that SCUBE1 is a surface protein predominantly expressed on MLL-r AML cells and high SCUBE1 expression is significantly associated with unfavorable prognosis of AML.

### Example 2—HOXA9 and MEIS1 Cooperatively Bind on Distal Regulatory Elements and Upregulate SCUBE1 Expression in MLL-r AML Cells

Translocations of MLL produce MLL oncofusion proteins that can activate transcription of downstream target genes, including the HOXA9 and MEIS1 transcription factors that functionally collaborate to drive leukemogenesis. SCUBE1 is highly expressed in MLL-r AML cells but not in normal hematopoietic stem/progenitor cells, peripheral blood cells or in leukemia cells lacking MLL-r. Hence, SCUBE1 might be directly regulated by MLL fusion genes such as MLL-AF9 or indirectly by its downstream homeodomain-containing transcription factor HOXA9 and its cofactor MEIS1, a member of the three-amino-acid-loop-extension protein family.

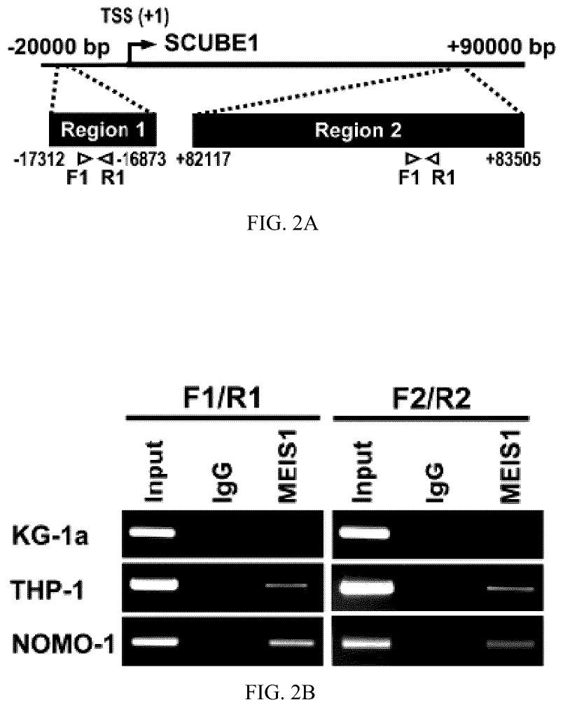

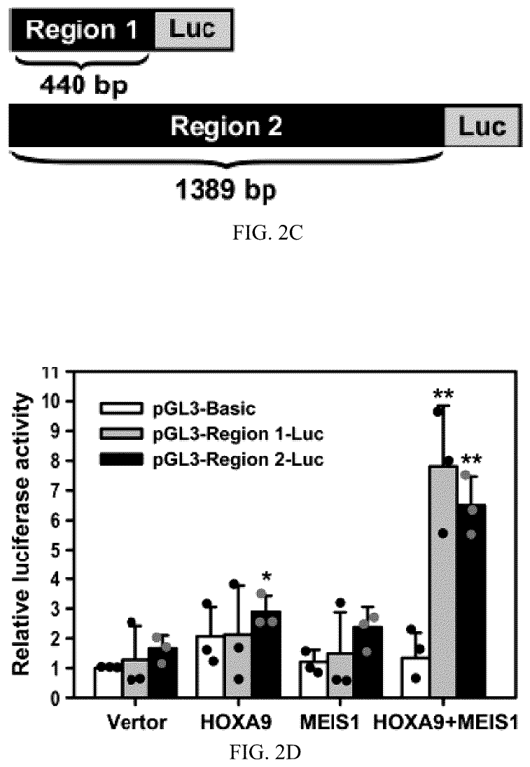

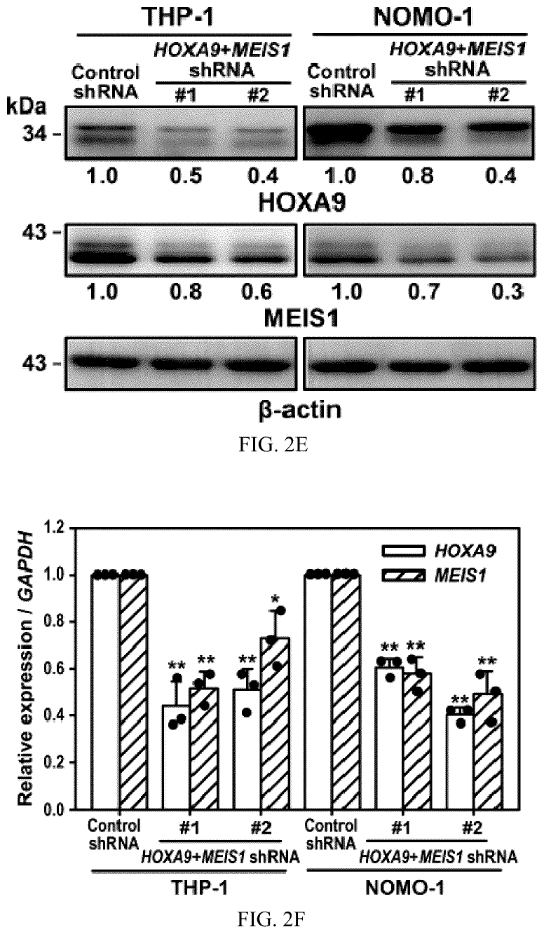

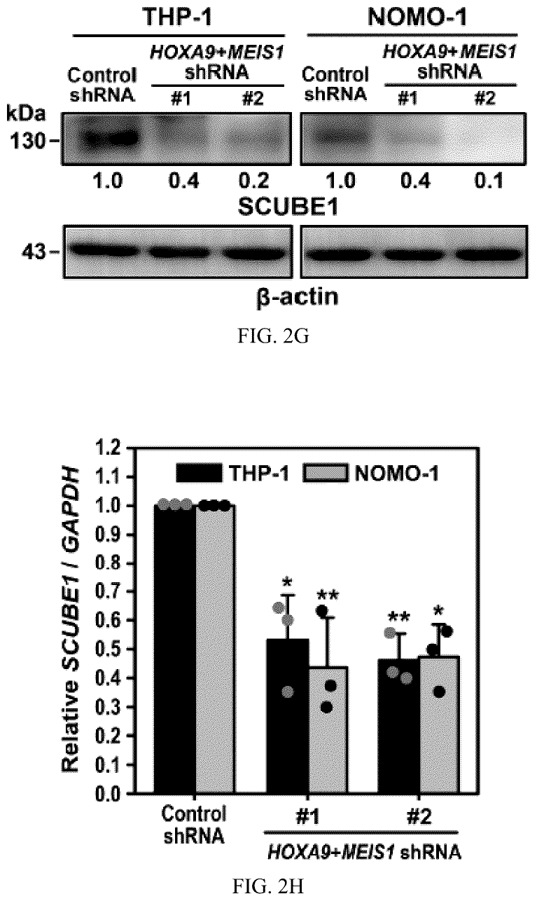

To determine whether MILL-AF9 fusion protein directly activates the SCUBE1 gene locus, we interrogated a previously published MLL-AF9 chromatin immunoprecipitation sequencing (ChIP-seq) dataset derived from THP-1 cells (Heiss E, Masson K, Sundberg C, et al. Identification of Y589 and Y599 in the juxtamembrane domain of Flt3 as ligand-induced autophosphorylation sites involved in binding of Src family kinases and the protein tyrosine phosphatase SHP2. Blood. 2006; 108(5):1542-50). However, virtually no peaks, evidenced by coincident signals in both MLL and MLL-AF9 fusion ChIP-seq tracks, localize within the SCUBE1 promoter region (data not shown). In addition, the gene body shows no significant enrichment of MLL-AF9-recruited epigenetic markers of H3K79me2, which further supports that SCUBE1 might not undergo transcriptional activation in MLL-r leukemia by directly targeting MLL-AF9. Rather, putative HOXA9/MEIS1 co-bound sites were found located in distal intergenic (20-kb upstream or 82-kb downstream) regulatory regions by an in silico bioinformatic tool PROMO. We then performed Chromatin Immunoprecipitation (ChIP) with an anti-MEIS1 antibody and confirmed that endogenous MEIS1 protein interacts with two distant regulatory DNA elements that harbor consensus HOXA9/MEIS1 co-bound sites in THP-1 and NOMO-1 cells (FIGS. 2A and B). In agreement with these findings, HOXA9 and MEIS1 cooperatively transactivate a regulatory DNA fragment containing the HOXA9/MEIS1 co-bound sites in a luciferase reporter assay (FIGS. 2C and D). Consistently, mutation of HOXA9/MEIS1 binding site abolishes HOXA9/MEIS1-mediated co-transactivation of luciferase reporter activity. Furthermore, double knockdown of HOXA9 and MEIS1 by two independent combinations of lentiviral-mediated delivery of short hairpin RNA (shRNA) (FIGS. 2E and F) significantly decreased the expression of SCUBE1 at both protein (FIG. 2G) and mRNA (FIG. 2H) levels in THP-1 or NOMO-1 cells. In agreement with previous genome-wide ChIP-seq experiments, our results suggest that SCUBE1 is likely a new target transactivated by cooperation between HOXA9 and MEIS1 at co-bound sites located in distal regulatory regions.

### Example 3—SCUBE1 is Required for In Vitro and In Vivo MLL-r Leukemia Cell Survival

To evaluate the functional role of SCUBE1 in MLL-r leukemia, we transduced THP-1, NOMO-1, and KG-1a leukemic cell lines with inducible lentiviral (shRNA) vectors targeting SCUBE1. After SCUBE1 depletion, cell growth was significantly reduced in MLL-r cell lines (THP-1 or NOMO-1), but growth was unaffected in KG-1a cells (a non-MLL-r leukemic cell line). Consistently, SCUBE1 knockdown led to disruption of the G1/S and G2/M phases of cell cycle progression, along with the induction of apoptosis in MLL-r leukemia cells, as revealed by a significant increase in cleaved caspase-3 and marked reduction of survivin. Both disruption of cell cycle progression and induction of apoptosis might contribute to the growth inhibitory effects of SCUBE1 knockdown in MLL-r leukemia cells.

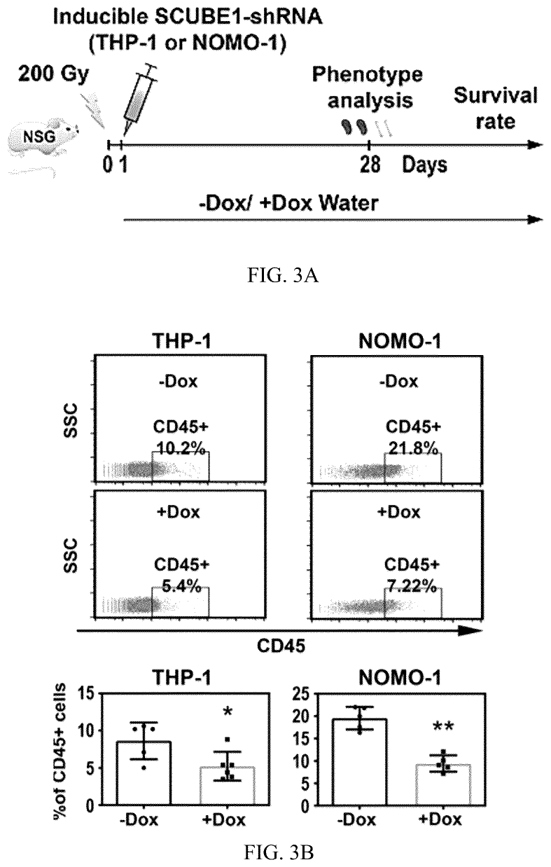

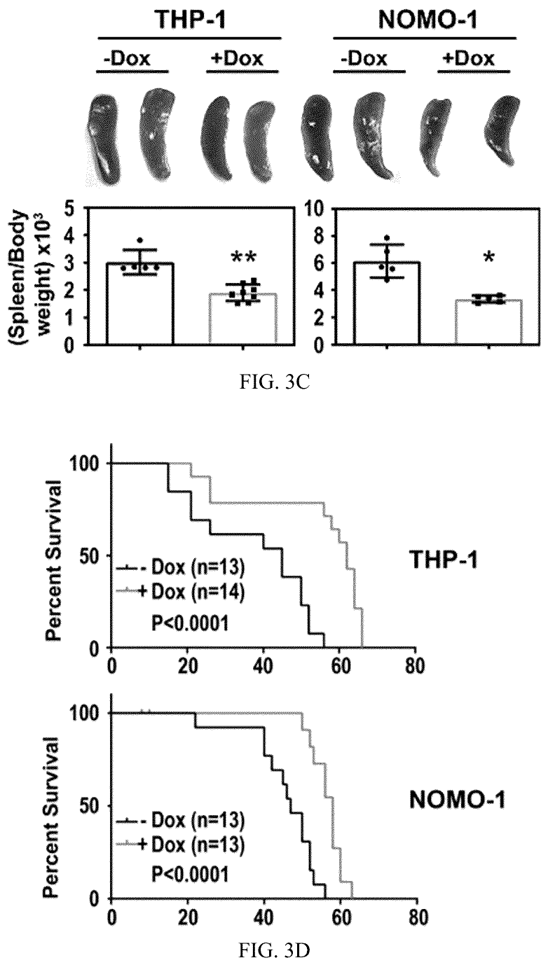

We next determined the role of SCUBE1 in leukemia propagation in vivo. THP-1 or NOMO-1 cells transduced with an inducible lentiviral SCUBE1 shRNA #2 vector were transplanted into NOD-Prkdc^{scid} Il2rg^{null} (NSG) mice (FIG. 3A). After treatment with Dox (+Dox) to induce SCUBE1 knockdown, mice transplanted with SCUBE1 shRNA #2 in THP-1 or NOMO-1 cells showed significant down-regulation of SCUBE1 expression, reduced engraftment in bone marrow (FIG. 3B) as well as reduced splenomegaly as compared with mice that did not receive Dox treatment (−Dox) (FIG. 3C). Importantly, knockdown of SCUBE1 (+Dox) significantly extended the survival of NSG mice as compared with control (Dox) mice (FIG. 3D). These data demonstrate a critical role for SCUBE1 in the growth and survival of MLL-AF9 leukemia cells both in vitro and in vivo.

### Example 4—SCUBE1 is Important for MLL-AF9-Induced Transformation In Vitro and MLL-AF9-Induced Leukemia Progression In Vivo

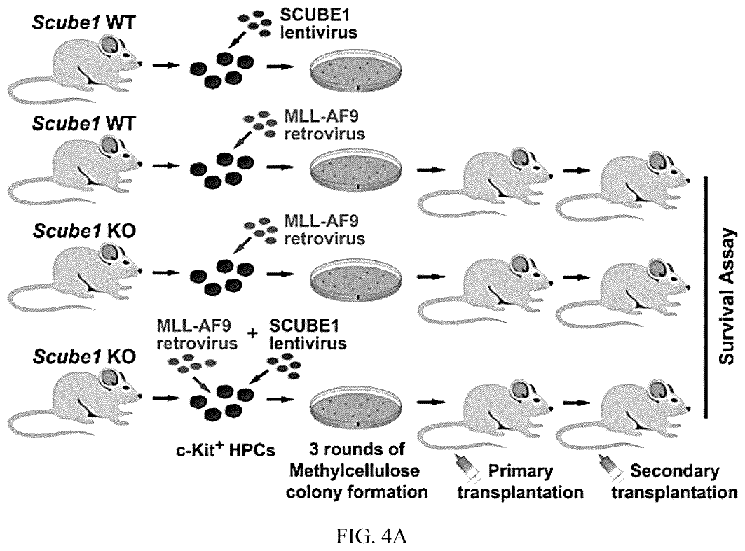

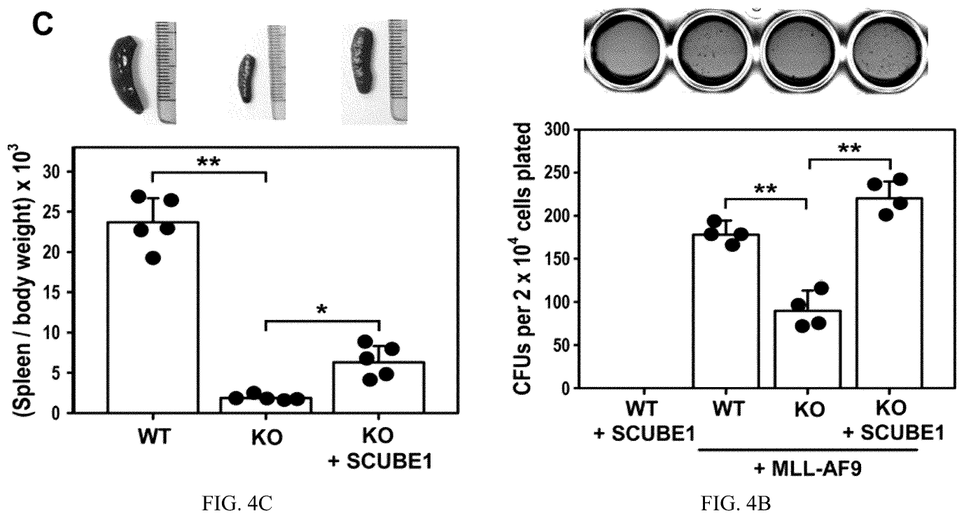

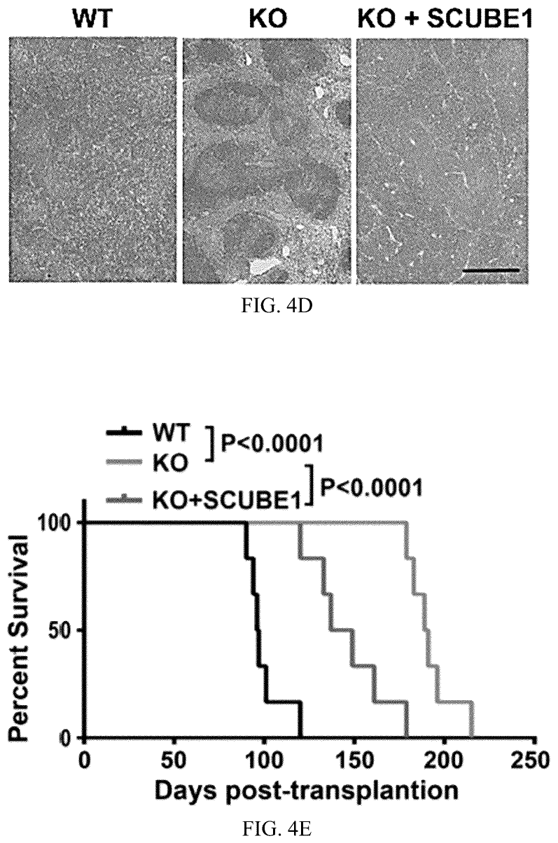

To further examine a role for SCUBE1 in leukemogenesis in vivo, we generated a new germline Scube1 knockout (KO) mutant mouse strain, Δ3. We first investigated the role of SCUBE1 in MLL-AF9-mediated transformation of hematopoietic progenitor cells (HPCs). c-Kit⁺ HPCs isolated from bone marrow of wild-type (WT) or KO mice were transduced with lentiviruses expressing SCUBE1 and/or retroviruses expressing MLL-AF9 as indicated (FIG. 4A). Of note, similar to human MLL-AF9 AML cells, MLL-AF9-mediated transformation of murine WT HPCs also markedly upregulated the cell surface expression of SCUBE1, which was not seen in KO cells. To assess the effect of Scube1 inactivation on MLL-AF9-mediated transformation, infected WT or KO cells were plated in methylcellulose. The number of viable colonies was reduced in the third round of methylcellulose replating in Scube1-KO versus WT HPCs (FIG. 4B). SCUBE1 overexpression alone did not drive the oncogenic transformation of the WT HPCs, whereas re-expression of SCUBE1 completely rescued the compromised MLL-AF9-mediated transformation by increasing the colony numbers in KO HPCs, like infected WT HPCs (FIG. 4B).

To investigate the importance of SCUBE1 in the progression of MLL-AF9-induced leukemia in vivo, donor MLL-AF9-transformed WT, KO, or KO HPCs with restoration of SCUBE1 expression (KO+SCUBE1) were serially transplanted into recipient C57BL/6J mice (FIG. 4A). All mice receiving WT MLL-AF9 transplantation died by 120 days after the second bone-marrow transplantation, whereas engraftment of KO MLL-AF9 cells (deletion of Scube1) markedly prolonged the survival of mice for more than 200 days (FIG. 4E). Consistently, re-expression of SCUBE1 (KO+SCUBE1) conferred a leukemic burden, leading to shorter survival. In agreement with the improved survival, SCUBE1 inactivation (KO) prevented splenomegaly (FIG. 4C) and reduced leukemia infiltration, resulting in normal spleen histology with a clear structure of red and white pulp as well as normal cell density in mice that received KO MLL-AF9 transplantation (FIG. 4D). By contrast, mice transplanted with WT or KO+SCUBE1 MLL-AF9 cells displayed profound leukemic blast infiltration and spleen hypercellularity (FIG. 4D). These results demonstrate a critical function of SCUBE1 in the initiation of MLL-AF9 leukemia in vitro as well as its progression in vivo.

### Example 5—SCUBE1 is Critical for Maintaining MLL-AF9 Transformation

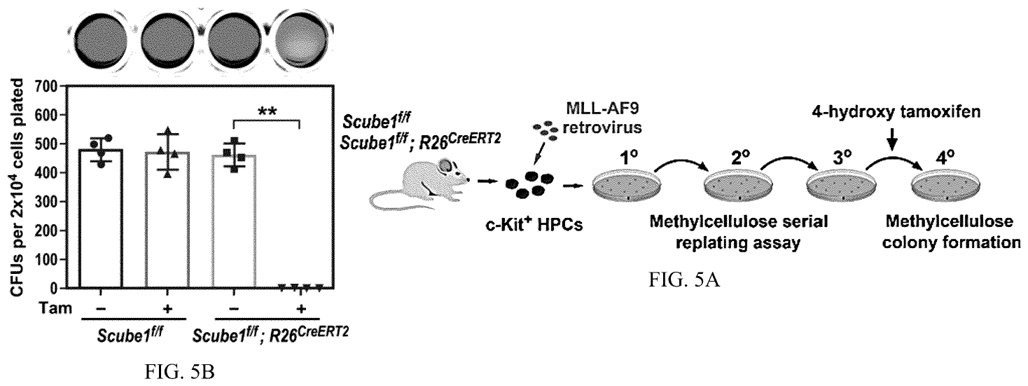

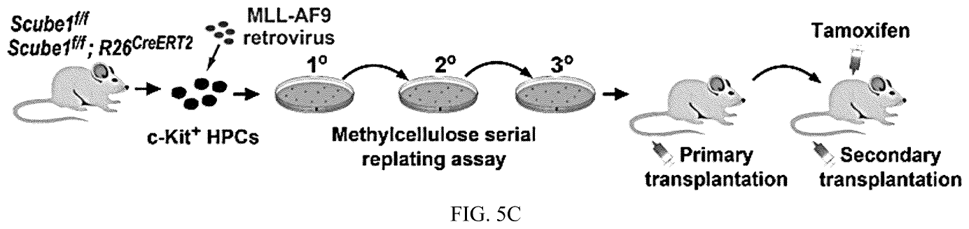

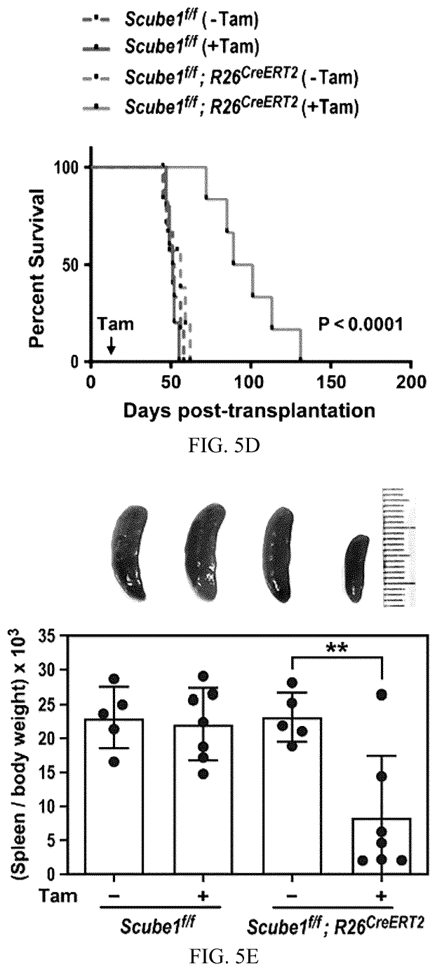

In addition to its function in initiating leukemia, SCUBE1 may also be required for maintaining the immortalized state elicited by MLL-AF9. To test this hypothesis, we used a tamoxifen-dependent conditional KO mouse model. Scube1 conditional KO (Scube1^{f/f}; R26^{CreERT2}) MLL-AF9-transformed HPCs failed to form colonies after 4-OHT-induced deletion of Scube1. By contrast, a similar treatment had no effect on control (Scube1^{f/f}) MLL-AF9-immortalized cells, which indicates that treatment with 4-OHT did not cause general cell toxicity (FIGS. 5A and B).

To further assess the in vivo effect of acute inactivation of Scube1 on leukemia maintenance, primary leukemias generated by transducing c-Kit⁺ HPCs from control or inducible KO mice with MLL-AF9 DsRed were transplanted into secondary recipient mice. When engraftment of leukemia cells reached 10% to 20% DsRed⁺ in peripheral blood cells, we daily administered tamoxifen (Tam) for 5 days to the secondary recipient mice (FIG. 5C). Effective deletion of Scube1 was verified by genotyping of peripheral blood cells at 2 weeks after Tam administration. In line with a critical role of SCUBE1 in maintaining the clonogenicity of the leukemic cells, acute Tam-induced Scube1 depletion significantly prolonged survival (FIG. 5D) and prevented splenomegaly (FIG. 5E) of Scube1^{f/f}; R26^{CreERT2} mice as compared with Scube1^{f/f} controls. In addition, apoptotic TUNEL (terminal deoxynucleotidyl transferase-mediated dUTP nick-end labeling) assay or immunostaining for Ki-67 (a proliferation marker) showed significantly more apoptotic cells and a markedly reduced number of proliferating MLL-AF9-induced leukemia stem cells (LSCs) in Scube1-KO than WT spleens, supporting that Scube1-KO indeed promotes apoptosis and suppresses proliferation of MLL-AF9-induced LSCs in vivo. Together, these data suggest that MLL-AF9-transformed HPCs require SCUBE1 for maintaining clonal growth both in vitro and in vivo.

### Example 6—Membrane SCUBE1 Binds FLT3 Ligand and FLT3 Receptor to Facilitate Activation of the FLT3-LYN Signaling Axis

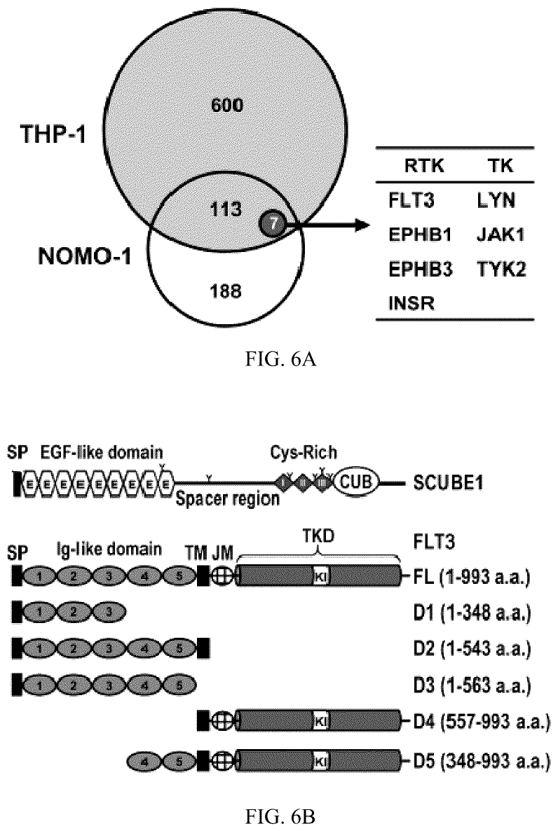

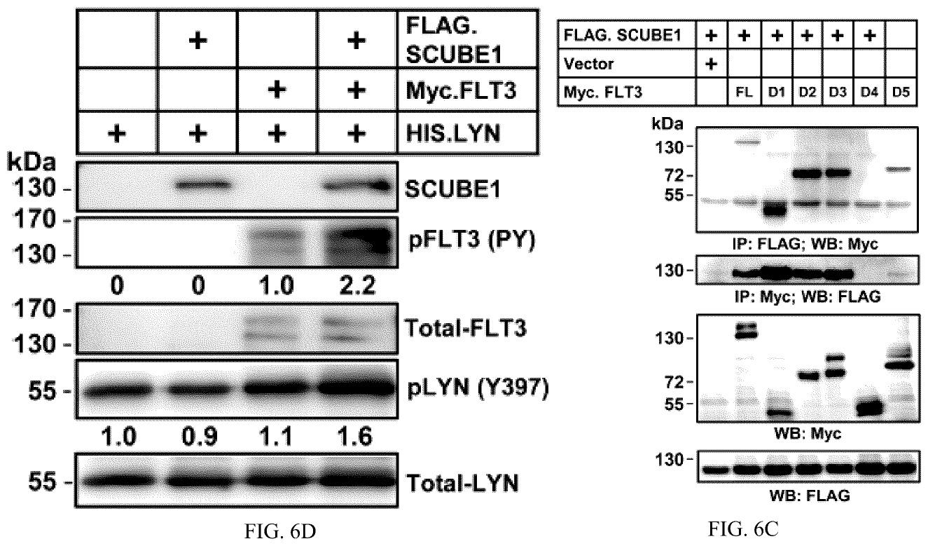

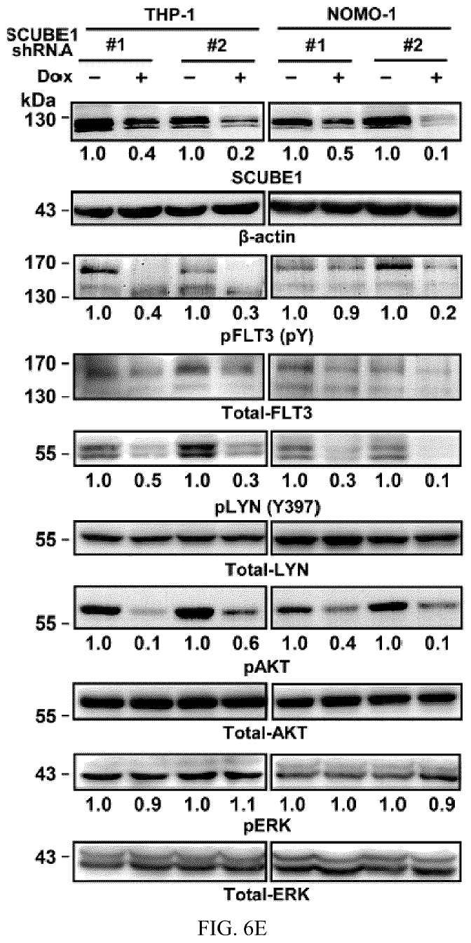

To further elucidate the molecular mechanisms underlying the contribution of membrane SCUBE1 to leukemogenesis, we used a proteomic proximity labeling assay31 to identify membrane proteins in the immediate vicinity of surface SCUBE1. We conjugated biotin to proteins proximal to SCUBE1 and analyzed the biotin labelled proteins by mass spectrometry. After excluding non-specific proteins, we identified a total of 120 membrane proteins associated with or in close proximity to SCUBE1 commonly shared in both THP-1 and NOMO-1 cells (FIG. 6A). Because SCUBE2 or SCUBE3 act as co-receptors to augment the signaling activity of receptor tyrosine kinases (RTKs) such as VEGFR or FGFR and because upregulation of protein-tyrosine kinase signaling is a hallmark of AML, we paid particular attention to RTKs and their downstream signaling components. Among the 120 identified proteins were; 4 RTKs [symbol] Fms-like receptor tyrosine kinase 3 (FLT3)), ephrin type-B receptor 1 and 3 (EPHB1 and 3), and insulin receptor (INSR) [symbol] as well as 3 nonreceptor TKs [symbol] Lck/Yes-related novel protein tyrosine kinase (LYN), Janus kinase 1 (JAK1) and tyrosine kinase 2 (TYK2) (FIG. 6A). FLT3, a class III RTK, consists of 5 extracellular ligand-binding Ig-like motifs, a member-spanning region, a juxtamembrane region followed by a TK domain interrupted by a kinase insert, and the carboxy terminal tail (FIG. 6B). FLT3 signaling is initiated by the binding of FLT3 ligand (FLT3L) to the extracellular Ig-like domains of FLT3 to induce dimerization, autophosphorylation, proximal recruitment of the Src family of non-receptor TKs such as LYN to be activated via tyrosine phosphorylation, and subsequent activation of downstream signaling pathways including phosphatidylinositol 3-kinase/AKT or extracellular signal-regulated kinases (ERKs).

Because the genes encoding FLT333 or its direct signaling component LYN are often over-expressed or mutated, thus leading to augmented proliferation and survival of human AML, we further examined whether SCUBE1 biochemically interacts with FLT3L or FLT3 and if so, whether SCUBE1 can modulate the signaling activity of the FLT3-LYN axis. HEK-293T cells were transfected with a FLAG-tagged SCUBE1 expression plasmid alone or with an expression plasmid encoding Myc-tagged FLT3 or His-tagged FLT3L. Immunoprecipitation with anti-FLAG antibody resulted in specific co-immunoprecipitation of FLT3 (FIG. 6C) or FLT3L. Further deletion mapping revealed that SCUBE1 primarily interacts with the ligand-binding extracellular Ig-like domains of FLT339 (FIG. 6C) or FLT3L via its spacer region and the CUB domain. Furthermore, SCUBE1 could interact and colocalize with endogenous FLT3 on the plasma membranes of THP-1 or NOMO-1 cells. Together, SCUBE1 might form a complex with FLT3L and FLT3 in MLL-r AML cells.

We further evaluated the effect of SCUBE1 on activating the FLT3-LYN signaling axis by reconstituting FLT3 and LYN expression in the absence or presence of SCUBE1 in HEK-293T cells. The tyrosine phosphorylation (pY) status of FLT3 (pFLT3) or LYN (pLYN) was measured by a pan or specific anti-pLYN (pY397) antibody. As shown in FIG. 6D, pFLT3 co-expressed with LYN showed a modest increase in expression, probably because of low expression of FLT3L in HEK-293T cells (https://www.proteinatlas.org), whereas ectopic expression of SCUBE1 markedly augmented pFLT3 as well as pLYN levels. Consistently, knockdown of SCUBE1 markedly decreased the intrinsic signaling activity of FLT3-LYN as well as the downstream activation of AKT (but not ERK), as reflected by decreased pY levels of these signaling components in THP-1 and NOMO-1 cells (FIG. 6E). In line with these findings, down-regulation of Flt3 phosphorylation was also observed in Scube1-knockout MLL-AF9 murine AML cells. Furthermore, SCUBE1-mediated specific tyrosine phosphorylation/activation of FLT3 slightly differed from that of FLT3L-induced FLT3 tyrosine phosphorylation (e.g., increased pY768 and pY842 but not pY591 level). Nevertheless, additional investigation is needed to fully elucidate the molecular mechanisms underlying the SCUBE1-assisted augmentation of FLT3 activation in AML cells. Together, these data suggest that membrane SCUBE1 might be a coreceptor to facilitate FLT3L binding to FLT3, thus promoting downstream LYN and AKT signaling.

### Example 7—SCUBE1-Targeting Antibody-Drug Conjugate (ADC) Effectively Inhibits Cancer Growth

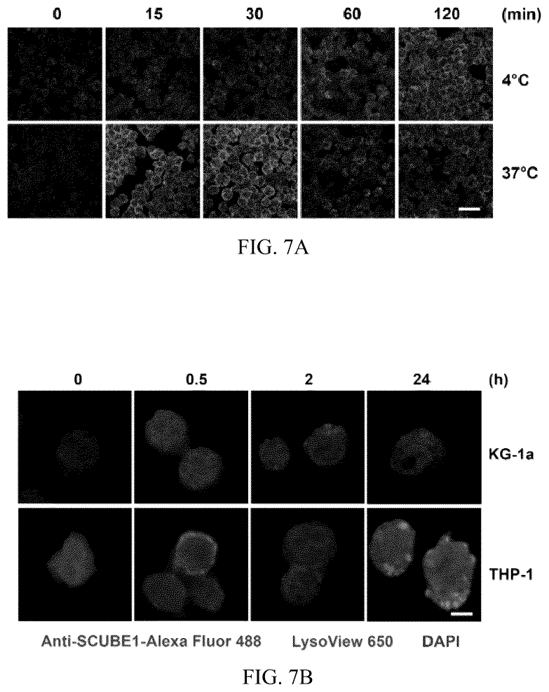

Internalization and trafficking to lysosomes upon antibody binding to a membrane target is a key mechanism for ADCs to exert their killing effect following intracellular release of cytotoxic payloads. We therefore examined whether a newly generated anti-SCUBE1 monoclonal antibody (mAb) clone #1 could internalize upon binding to SCUBE1 on leukemia cells. As shown in FIGS. 7A, we incubated the anti-SCUBE1 antibody with THP-1 cells and found that the mAb rapidly bound, efficiently endocytosed to lysosomes and degraded after 24 h, which suggests that this mAb can be internalized. Furthermore, as shown in FIG. 7B, internalization of the mAb was observed in SCUBE1 positive cells (i.e., THP-1 cells) rather than SCUBE1 negative cells (i.e., KG-1a). These results demonstrate the potential of the anti-SCUBE1 monoclonal antibody (mAb) to specifically recognize malignant cells with high expression of SCUBE1 on the cell surface.

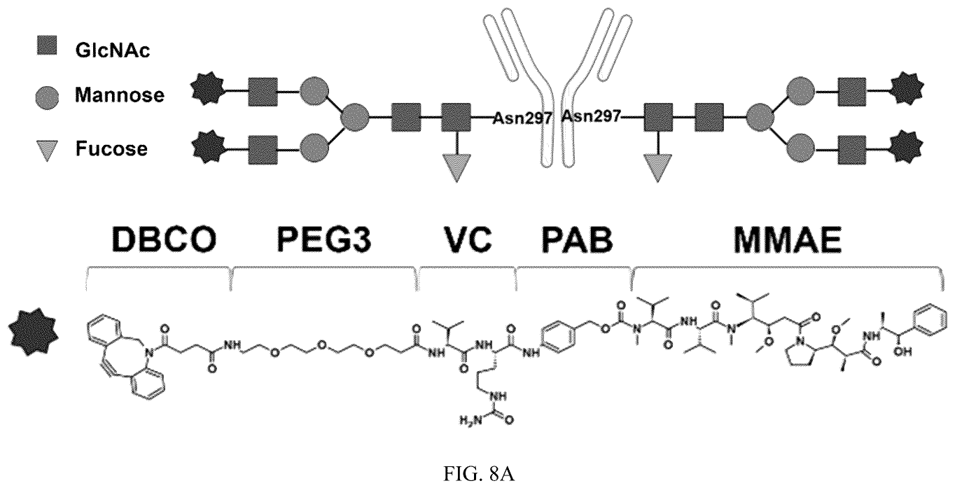

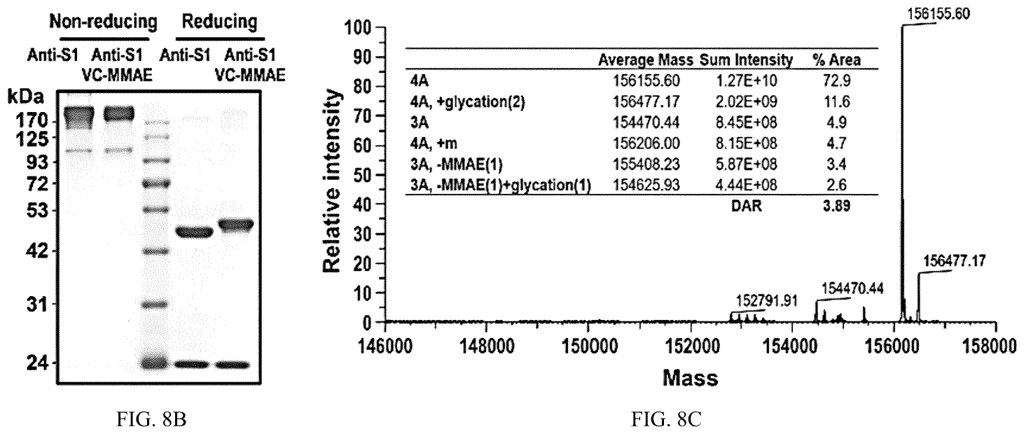

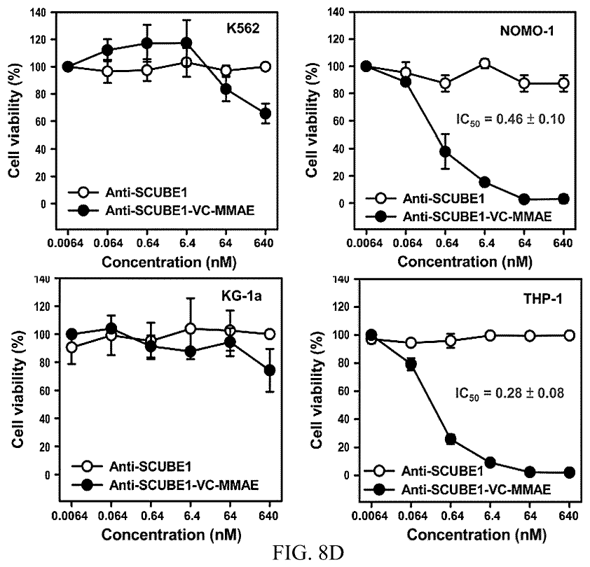

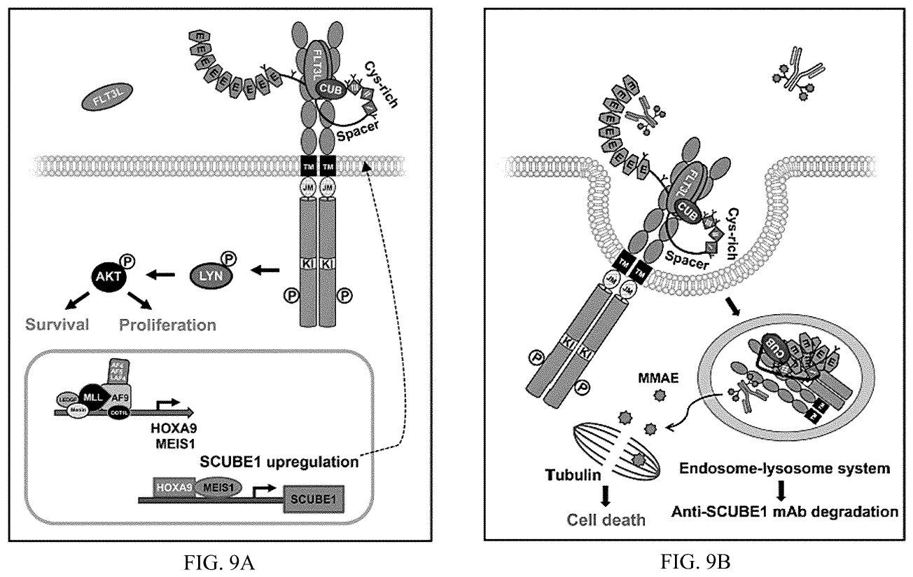

As a proof of concept for its potential therapeutic use, we generated an ADC combining mAb #1 as the SCUBE1-targeting moiety with a proteolytically cleavable valine-citrulline (VC) linker and the anti-microtubule cytotoxic agent monomethyl auristatin E (MMAE) (see FIG. 8A) by using the homogeneous trimannosyl glycoengineering platform as described in WO 2018/126092 A1, the entirety of which is incorporated by reference. The average drug-to-antibody ratio (DAR) was 3.89 (FIGS. 8B and 8C). Importantly, this ADC (designated as anti-SCUBE1-VC-MMAE) retained similar binding affinity as the parental antibody. Five-day treatment with this ADC was effective in reducing cell viability in SCUBE1-expressing MLL-r leukemia cell lines THP-1 and NOMO-1 (half maximal inhibitory concentration=0.28±0.08 and 0.46±0.1 nM, respectively), with no effect seen in SCUBE1-negative KG-1a and K562 cells (FIG. 8D) or normal murine HPCs. To further evaluate the efficacy of the ADC in vivo, we subcutaneously transplanted THP-1 cells into NSG mice. After treatment with anti-SCUBE1 ADC, THP-1 tumor growth was significantly reduced as compared with the IgG control. In addition, no antigen-independent toxicity was observed in either treatment group, as evaluated by monitoring body weight loss. These results confirm the selectivity of this ADC and suggest that surface SCUBE1 could be exploited as an MLL-r specific biomarker and could potentially be used as a therapeutic target (FIGS. 9A and 9B).

### Example 8—Anti-SCUBE1-VC-MMAE Reduced Tumor Growth of THP-1 Cells in a Subcutaneous Model

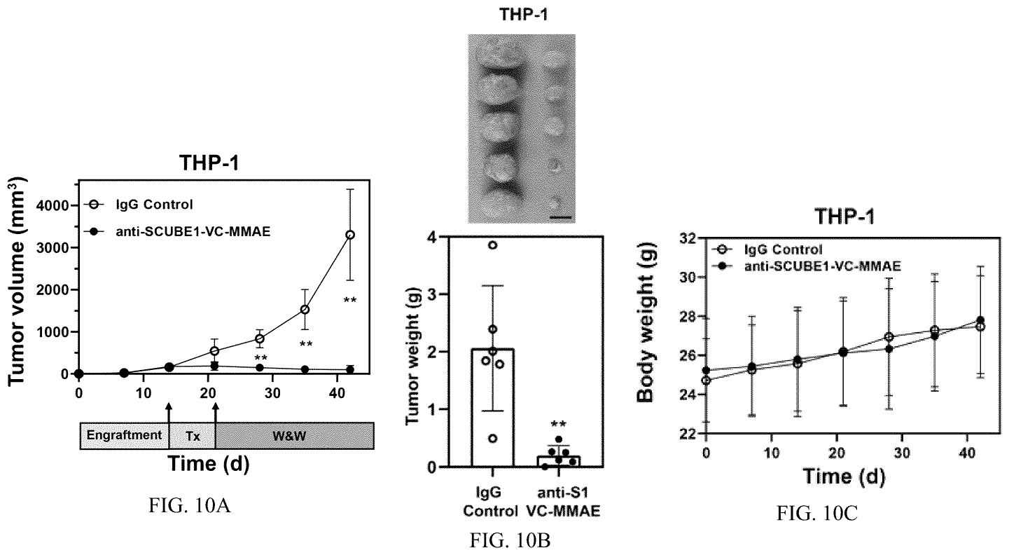

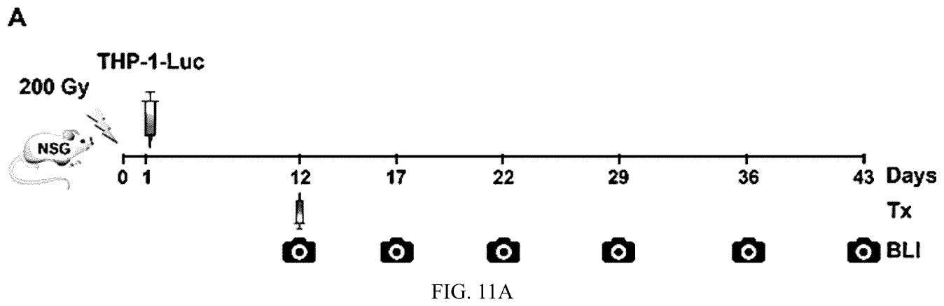

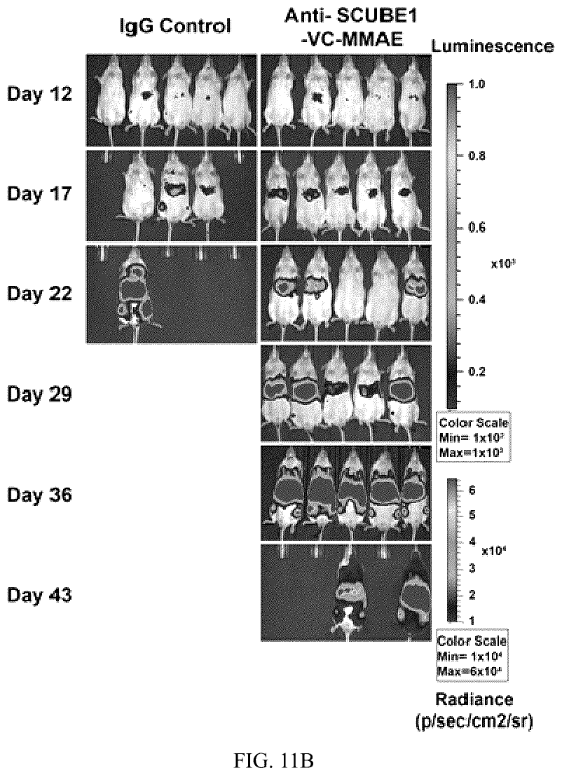

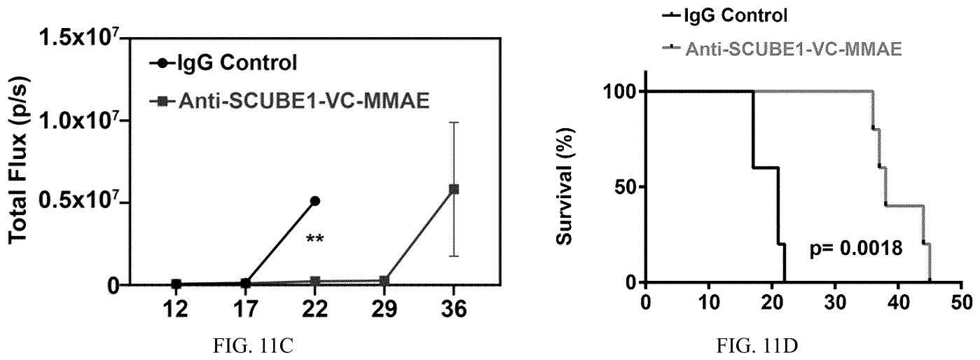

To explore in vivo efficacy of the anti-SCUBE1-VC-MMAE, THP-1 cells were subcutaneously injected in NSG mice. After palpable tumors were evident (tumor volume ˜150-200 mm³), mice were randomly assigned for treatment of 10 mg/kg human IgG or anti-SCUBE1 ADC. As shown in FIG. 10A, the drugs were administered intravenously for a total dose of 2 at 1 week apart as indicated by arrows (Tx). After the completion of treatment, tumor growth was monitored in a wait-and-watch (W&W) period. The tumor volume was measured by using a digital caliper. Mice were sacrificed once the tumor volume exceeded 2000 mm³ and tumor weight was measured after isolation.

After treatment with anti-SCUBE1-VC-MMAE, tumor growth in mice was significantly suppressed (FIGS. 10A and 10B). In addition, as shown in FIG. 10C, anti-SCUBE1-VC-MMAE was well-tolerated by the mice being treated with the same as evidenced by no significant weight change.

### Example 9—Anti-SCUBE1 ADC Reduced Leukemia Growth of THP-1 Cells and Enhanced Survival in Orthotopic Model

To further validate in vivo efficacy of the anti-SCUBE1 ADC in a more disease-relevant environment. THP-1 cells having stable luciferase expression (THP-1-Luc) were intravenously injected in sub-lethally irradiated NSG mice. As can be seen in FIG. 11A, the mice were randomly assigned for treatment of 5 mg/kg human IgG or anti-SCUBE1 ADC on day 12 after intravenous injection of THP-1 cells, and the drugs were administered intravenously for once as indicated by Tx. Leukemia burden was monitored weekly by bioluminescent imaging (BLI) using in vivo imaging solution (IVIS) and overall survival was monitored by leukemic symptom (hunch back, loss of mobility, rough coat, and/or hind limb paralysis).

As shown in FIGS. 11B and 11C, the anti-SCUBE1 ADC resulted in a prominent delay of detectable bioluminescence (IgG Control vs. Anti-SCUBE1 ADC: 17 vs. 29 days of maintaining non-detectable bioluminescence), suggesting that the leukemia growth of the THP-1 cells was reduced after the anti-SCUBE1 ADC treatment. Moreover, mice treated with the anti-SCUBE1 ADC had higher overall survival (FIG. 11C). These results further demonstrate in vivo efficacy of the anti-SCUBE1 ADC described herein.

What is claimed is:

1. An antibody or antigen-binding fragment thereof that specifically binds to SCUBE1, comprising a heavy chain variable region and/or a light chain variable region, wherein:
the heavy chain variable region comprises:
a complementary determining region (CDR) sequence CDRH1 comprising the amino acid sequence of GYTFTSYAMH (SEQ ID NO: 1) or an amino acid sequence having at least 80% sequence identity to SEQ ID NO: 1;
a CDRH2 sequence comprising the amino acid sequence of YINPYNDVSRYNEKFQG (SEQ ID NO: 2) or an amino acid sequence having at least 80% sequence identity to SEQ ID NO: 2; and
a CDRH3 sequence comprising the amino acid sequence of EARPTSAPYFDV (SEQ ID NO: 3) or an amino acid sequence having at least 80% sequence identity to SEQ ID NO: 3; and wherein:
the light chain variable region comprises:
a CDRL1 sequence comprising the amino acid sequence of KSSQSLLNSRTRKNYLA (SEQ ID NO: 4) or an amino acid sequence having at least 80% sequence identity to SEQ ID NO: 4;
a CDRL2 sequence comprising the amino acid sequence of WTSTRES (SEQ ID NO: 5) or an amino acid sequence having at least 80% sequence identity to SEQ ID NO: 5; and
a CDRL3 sequence comprising the amino acid sequence of KQSYNLFT (SEQ ID NO: 6) or an amino acid sequence having at least 80% sequence identity to SEQ ID NO: 6.

2. The antibody or antigen-binding fragment thereof according to claim 1, wherein:
the heavy chain variable region comprises the amino acid sequence of SEQ ID NO: 7 or an amino acid sequence having at least 80% sequence identity to SEQ ID NO: 7; and/or
the light chain variable region comprises the amino acid sequence of SEQ ID NO: 8 or an amino acid sequence having at least 80% sequence identity to SEQ ID NO: 8.

3. The antibody or antigen-binding fragment thereof according to claim 1, wherein the antibody is a monoclonal antibody, chimeric antibody, humanized antibody, or human antibody.

4. A vector encoding the antibody or antigen-binding fragment thereof of claim 1.

5. A genetically engineered cell expressing the antibody or antigen-binding fragment thereof as defined in claim 1.

6. A pharmaceutical composition comprising the antibody or antigen-binding fragment thereof as defined in claim 1 and a pharmaceutically acceptable carrier.

7. An antibody-drug conjugate (ADC) comprising an anti-SCUBE1 antibody, or the antigen-binding fragment thereof as defined in claim 1, conjugated to a cytotoxin.

8. The ADC of claim 7, wherein the ADC has the structure of the following formula:
Ab-(Z-L-D)ₙ,
wherein an antibody or antigen-binding fragment thereof (Ab) is conjugated (covalently linked) to a linker (L), through a chemical moiety (Z), and further to a cytotoxin moiety (“drug,” D); and wherein n represents the number of drugs linked to the antibody.

9. The ADC of claim 8, wherein n is from about 1 to about 20.

10. The ADC of claim 7, wherein the cytotoxin is a microtubule-binding agent (for instance, maytansine or a maytansinoid), an amatoxin, Pseudomonas exotoxin A, deBouganin, diphtheria toxin, saporin, an auristatin, an anthracycline, a calicheamicin, irinotecan, SN-38, a duocarmycin, a pyrrolobenzodiazepine, a pyrrolobenzodiazepine dimer, an indolinobenzodiazepine, an indolinobenzodiazepine dimer, or a variant thereof.

11. The ADC of claim 7, wherein the cytotoxin is a DNA-intercalating agents, (e.g., anthracyclines), agents capable of disrupting the mitotic spindle apparatus (e.g., Vinca alkaloids, maytansine, maytansinoids, and derivatives thereof), RNA polymerase inhibitors (e.g., an amatoxin, such as .alpha.-amanitin, and derivatives thereof), and agents capable of disrupting protein biosynthesis (e.g., agents that exhibit rRNA N-glycosidase activity, such as saporin and ricin A-chain).

12. The ADC of claim 7, wherein the cytotoxin is MMAE.

13. A pharmaceutical composition comprising the ADC of claim 7 and a pharmaceutically acceptable carrier.

14. A method of treating and/or preventing a SCUBE1-expressing cancer in a subject, said method comprising administering the ADC of claim 7 to the subject.

15. The method of claim 14, wherein the cancer is a blood cancer.

16. The method of claim 14, wherein the cancer is leukemia.

17. The method of claim 14, wherein the cancer is leukemia caused by MLL rearrangements.

18. The method of claim 14, wherein the cancer is AML.

19. The method of claim 14, wherein the cancer is MLL-r AML.

20. A genetically engineered cell comprising the vector of claim 4.
